SEQUENCE LISTING (111 sequences)

SEQ ID NO: 1 - DNA, 38 bp, artificial
  primer
gggcccggtaccatgtcctcagccatctatcccagcct

SEQ ID NO: 2 - DNA, 38 bp, artificial
  primer
gggcccggatccttaacgccagccggcgtcgatccagt

SEQ ID NO: 3 - DNA, 52 bp, artificial
  primer
gggcccggatccaggagaaattaactatgaccgctcaagtcacttgcgtatg

SEQ ID NO: 4 - DNA, 38 bp, artificial
  primer
gggcccgctagcttagacaaggcggacctcatgctggg

SEQ ID NO: 5 - DNA, 52 bp, artificial
  primer
gggcccgctagcaggagaaattaactatgaggtccgccttgtctaaccgcac

SEQ ID NO: 6 - DNA, 38 bp, artificial
  primer
gggcccaagcttttagtggttgtggcggggcagcttgg

SEQ ID NO: 7 - DNA, 52 bp, artificial
  primer
gggcccaagcttaggagaaattaactatggtttgtcggcggcttctagcatg

SEQ ID NO: 8 - DNA, 59 bp, artificial
  primer
gcgcagctggcgttgttgtccttggcctttctgagcagcagggccgaacgaccttcgaa

SEQ ID NO: 9 - DNA, 59 bp, artificial
  primer
ttcgaaggtcgttcggccctgctgctcagaaaggccaaggacaacaacgccagctgcgc

SEQ ID NO: 10 - DNA, 39 bp, artificial
  primer
gggcccgctcagcttagaggaggccgcggccggccaggt

SEQ ID NO: 11 - DNA, 38 bp, artificial
  primer
gggcccggtaccatgaccgacaccctgcgccattacat

SEQ ID NO: 12 - DNA, 38 bp, artificial
  primer
gggccctctagattacgaccacgagtaggaggttttgg

SEQ ID NO: 13 - DNA, 38 bp, artificial
  primer
gggcccggtaccatggcttcggtacacggcaccacata

SEQ ID NO: 14 - DNA, 38 bp, artificial
  primer
gggcccgcatgcttacttcaccgggcttacggtgctta

SEQ ID NO: 15 - DNA, 38 bp, artificial
  primer
gggcccggtaccatgtatacagtaggagattacctatt

SEQ ID NO: 16 - DNA, 38 bp, artificial
  primer
gggcccgcatgcttatgatttattttgttcagcaaata

SEQ ID NO: 17 - DNA, 38 bp, artificial
  primer
gggcccggtaccatgcaaaacccctataccgtggccga

SEQ ID NO: 18 - DNA, 38 bp, artificial
  primer
gggcccgcatgcttatcccccgttgcgggcttccagcg

SEQ ID NO: 19 - DNA, 52 bp, artificial
  primer
gggcccgcatgcaggagaaattaactatgaacaactttaatctgcacacccc

SEQ ID NO: 20 - DNA, 38 bp, artificial
  primer
gggccctctagattagcgggcggcttcgtatatacggc

SEQ ID NO: 21 - DNA, 50 bp, artificial
  primer
gggcccgcatgcaggagatataccatgtcttatcctgagaaatttgaagg

SEQ ID NO: 22 - DNA, 38 bp, artificial
  primer
gggccctctagactagtctgaaaattctttgtcgtagc

SEQ ID NO: 23 - DNA, 51 bp, artificial
  primer
gggcccgcatgcaaggagatataccatgaagatcaaagctgttggtgcata

SEQ ID NO: 24 - DNA, 38 bp, artificial
  primer
gggccctctagattagtctgttagtgtgcgattatcga

SEQ ID NO: 25 - DNA, 51 bp, artificial
  primer
gggcccgcatgcaaggagatataccatgtcgatgataaaaagctatgccgc

SEQ ID NO: 26 - DNA, 38 bp, artificial
  primer
gggccctctagattaaaaatcggctttcaacaccacgc

SEQ ID NO: 27 - DNA, 51 bp, artificial
  primer
gggcccgcatgcaaggagatataccatgaaagcagcagtagtaagacacaa

SEQ ID NO: 28 - DNA, 38 bp, artificial
  primer
gggccctctagattatttagtaaaatcaatgaccattc

SEQ ID NO: 29 - DNA, 43 bp, artificial
  primer
gggcccggtctcaatagtttctcctctttaatgaattcggtca

SEQ ID NO: 30 - DNA, 49 bp, artificial
  primer
gggcccggtctcactatggtaccatgagccgtatcagaaaagcacccgc

SEQ ID NO: 31 - DNA, 43 bp, artificial
  primer
gggcccggtctcattacttaaaccgtcgcggcttttttcggaa

SEQ ID NO: 32 - DNA, 60 bp, artificial
  primer
gggcccggtctcagtaagctagcaggagaaattaactatggccaacatcgaaatcagaca

SEQ ID NO: 33 - DNA, 39 bp, artificial
  primer
gggcccggtctcaaccgccatcaggccgtacggcgtacc

SEQ ID NO: 34 - DNA, 41 bp, artificial
  primer
gggcccggtctcacggtgcccgtcattaaaatggcaacccg

SEQ ID NO: 35 - DNA, 43 bp, artificial
  primer
gggcccggtctcacaggttaggtcaccggtgccgggttaaaca

SEQ ID NO: 36 - DNA, 60 bp, artificial
  primer
gggcccggtctcacctgaagcttaggagaaattaactatgaccactacccccttcaatcg

SEQ ID NO: 37 - DNA, 41 bp, artificial
  primer
gggcccggtctcatgtctcgatgccgtagcggtcaaagtag

SEQ ID NO: 38 - DNA, 41 bp, artificial
  primer
gggcccggtctcagacagtcagcattcgcatcggctcgtcg

SEQ ID NO: 39 - DNA, 43 bp, artificial
  primer
gggcccggtctcagcggttagttgaacgggccggccacggcga

SEQ ID NO: 40 - DNA, 50 bp, artificial
  primer
gggcccggtctcaccgcgctgagctctagaggcatcaaataaaacgaaag

SEQ ID NO: 41 - DNA, 58 bp, artificial
  primer
tttaaatgatgagtgaaggctcctgttgagtagtctgtgagtttaactccaagcatca

SEQ ID NO: 42 - DNA, 58 bp, artificial
  primer
tgatgcttggagttaaactcacagactactcaacaggagccttcactcatcatttaaa

SEQ ID NO: 43 - DNA, 58 bp, artificial
  primer
taaatgatgagtgaaggctcctgttgagaggtctgtgagtttaactccaagcatcagg

SEQ ID NO: 44 - DNA, 58 bp, artificial
  primer
cctgatgcttggagttaaactcacagacctctcaacaggagccttcactcatcattta

SEQ ID NO: 45 - DNA, 58 bp, artificial
  primer
ttaaatgatgagtgaaggctcctgttgagaagtctgtgagtttaactccaagcatcag

SEQ ID NO: 46 - DNA, 58 bp, artificial
  primer
ctgatgcttggagttaaactcacagacttctcaacaggagccttcactcatcatttaa

SEQ ID NO: 47 - DNA, 59 bp, artificial
  primer
ttgatttggtccatgaatttctctttcgattgtataaccatcattattgataataaagc

SEQ ID NO: 48 - DNA, 59 bp, artificial
  primer
gctttattatcaataatgatggttatacaatcgaaagagaaattcatggaccaaatcaa

SEQ ID NO: 49 - DNA, 59 bp, artificial
  primer
ttgatttggtccatgaatttctctttcgagtgtataaccatcattattgataataaagc

SEQ ID NO: 50 - DNA, 59 bp, artificial
  primer
gctttattatcaataatgatggttatacactcgaaagagaaattcatggaccaaatcaa

SEQ ID NO: 51 - DNA, 55 bp, artificial
  primer
tgatttggtccatgaatttctctttccattgtataaccatcattattgataataa

SEQ ID NO: 52 - DNA, 55 bp, artificial
  primer
ttattatcaataatgatggttatacaatggaaagagaaattcatggaccaaatca

SEQ ID NO: 53 - DNA, 55 bp, artificial
  primer
tcattgtagctttgatttggtccatggaattctctttcgactgtataaccatcat

SEQ ID NO: 54 - DNA, 55 bp, artificial
  primer
atgatggttatacagtcgaaagagaattccatggaccaaatcaaagctacaatga

SEQ ID NO: 55 - DNA, 55 bp, artificial
  primer
tcattgtagctttgatttggtccatggtgttctctttcgactgtataaccatcat

SEQ ID NO: 56 - DNA, 55 bp, artificial
  primer
atgatggttatacagtcgaaagagaacaccatggaccaaatcaaagctacaatga

SEQ ID NO: 57 - DNA, 55 bp, artificial
  primer
tcattgtagctttgatttggtccatgcagttctctttcgactgtataaccatcat

SEQ ID NO: 58 - DNA, 55 bp, artificial
  primer
atgatggttatacagtcgaaagagaactgcatggaccaaatcaaagctacaatga

SEQ ID NO: 59 - DNA, 55 bp, artificial
  primer
tcattgtagctttgatttggtccatgcatttctctttcgactgtataaccatcat

SEQ ID NO: 60 - DNA, 55 bp, artificial
  primer
atgatggttatacagtcgaaagagaaatgcatggaccaaatcaaagctacaatga

SEQ ID NO: 61 - DNA, 38 bp, artificial
  primer
gggcccggatccatgtatacagtaggagattacctatt

SEQ ID NO: 62 - DNA, 38 bp, artificial
  primer
gggccctctagattatgatttattttgttcagcaaata

SEQ ID NO: 63 - DNA, 38 bp, artificial
  primer
gggcccggatccatgaacaactttaatctgcacacccc

SEQ ID NO: 64 - DNA, 38 bp, artificial; identical to SEQ ID NO: 20
  primer

SEQ ID NO: 65 - DNA, 38 bp, artificial
  primer
gggcccggatccatgaccgacaccctgcgccattacat

SEQ ID NO: 66 - DNA, 38 bp, artificial; identical to SEQ ID NO: 12
  primer

SEQ ID NO: 67 - DNA, 38 bp, artificial
  primer
gggcccggatccatggtttgtcggcggcttctagcatg

SEQ ID NO: 68 - DNA, 38 bp, artificial
  primer
gggccctctagattagaggaggccgcggccggccaggt

SEQ ID NO: 69 - DNA, 59 bp, artificial
  primer
ccgaattcattaaagaggagaaaggtaccatgtcggcaacgaaacccaggctgcgctcc

SEQ ID NO: 70 - DNA, 60 bp, artificial
  primer
gatcctgcgtcagtcaaacggcgggctagctcagtgcgagtggctcggcacctccgcgcc

SEQ ID NO: 71 - DNA, 60 bp, artificial
  primer
gaggtgccgagccactcgcactgagctagcccgccgtttgactgacgcaggatccgaacc

SEQ ID NO: 72 - DNA, 60 bp, artificial
  primer
ggctcatcgtgcgctccttggttcgttgctcaccgtgcccagcgcagcacgagcggatcg

SEQ ID NO: 73 - DNA, 60 bp, artificial
  primer
cgatccgctcgtgctgcgctgggcacggtgagcaacgaaccaaggagcgcacgatgagcc

SEQ ID NO: 74 - DNA, 60 bp, artificial
  primer
tcgatgctcaggcggcgcgcacgcaagctttcagcggccgaacgcttcggtgtcgacgcg

SEQ ID NO: 75 - DNA, 60 bp, artificial
  primer
gacaccgaagcgttcggccgctgaaagcttgcgtgcgcgccgcctgagcatcgattatcg

SEQ ID NO: 76 - DNA, 60 bp, artificial
  primer
ttgcgccgcgtcgccgccatatgtcaggttccgacgccgcgcttcagtgcgaatcgcgcg

SEQ ID NO: 77 - DNA, 60 bp, artificial
  primer
ctgaagcgcggcgtcggaacctgacatatggcggcgacgcggcgcaacccgacctgggcc

SEQ ID NO: 78 - DNA, 60 bp, artificial
  primer
tcgttttatttgatgcctctagagctcagctcagatcgggtaatgccgcggcgcggtctg

SEQ ID NO: 79 - DNA, 60 bp, artificial
  primer
ccaggtcgggttgcgccgcgtcgccgcgctcagctcaggttccgacgccgcgcttcagtg

SEQ ID NO: 80 - DNA, 52 bp, artificial
  primer
aattcattaaagaggagaaaggtaccatgtcgtacgcaatctatcccagcct

SEQ ID NO: 81 - DNA, 60 bp, artificial
  primer
acagggggatgaattttcatagttaatttctcctggatccttattctccgtaccacccgg

SEQ ID NO: 82 - DNA, 60 bp, artificial
  primer
ccgggtggtacggagaataaggatccaggagaaattaactatgaaaattcatccccctgt

SEQ ID NO: 83 - DNA, 60 bp, artificial
  primer
cgcggtgtggatgctgacatagttaatttctcctgctagcttattgcgcgaagccccatt

SEQ ID NO: 84 - DNA, 60 bp, artificial
  primer
aatggggcttcgcgcaataagctagcaggagaaattaactatgtcagcatccacaccgcg

SEQ ID NO: 85 - DNA, 60 bp, artificial
  primer
gatggagaagttgcggacatagttaatttctcctaagcttttagtgcgaatgcctcggat

SEQ ID NO: 86 - DNA, 60 bp, artificial
  primer
atccgaggcattcgcactaaaagcttaggagaaattaactatgtccgcaacttctccatc

SEQ ID NO: 87 - DNA, 56 bp, artificial
  primer
cgttttatttgatgcctctagacatatgttaggccgacgcaagcagcccgcgtgcg

SEQ ID NO: 88 - DNA, 40 bp, artificial
  primer
ttaaagaggagaaaggtaccatgagccagtttgcgaacta

SEQ ID NO: 89 - DNA, 40 bp, artificial
  primer
ttttatttgatgcctctagattaaaccgcgcccggactca

SEQ ID NO: 90 - DNA, 50 bp, artificial
  primer
gcatcaccatcaccatcacggatccatgtcgtacgcaatctatcccagcc

SEQ ID NO: 91 - DNA, 50 bp, artificial
  primer
tttcgttttatttgatgcctctagattattctccgtaccacccggcgtcg

SEQ ID NO: 92 - DNA, 50 bp, artificial
  primer
gcatcaccatcaccatcacggatccatgtcagcatccacaccgcgccggc

SEQ ID NO: 93 - DNA, 50 bp, artificial
  primer
tttcgttttatttgatgcctctagattagtgcgaatgcctcggattgccg

SEQ ID NO: 94 - DNA, 50 bp, artificial
  primer
gcatcaccatcaccatcacggatccatgtccgcaacttctccatccagtt

SEQ ID NO: 95 - DNA, 50 bp, artificial
  primer
tttcgttttatttgatgcctctagattaggccgacgcaagcagcccgcgt

SEQ ID NO: 96 - DNA, 60 bp, artificial
  primer
actgaccgaattcattaaagaggagaaaggtaccatgtcggcaacaaaacccaggctgcg

SEQ ID NO: 97 - DNA, 60 bp, artificial
  primer
ctgctcgatgtcatagttaatttctcctgctagctcaatgcgaatggctcggcacgtccg

SEQ ID NO: 98 - DNA, 60 bp, artificial
  primer
gccattcgcattgagctagcaggagaaattaactatgacatcgagcagcacaccgcgcta

SEQ ID NO: 99 - DNA, 60 bp, artificial
  primer
caatctgttgcatgggttttttctcctgaagctttcagcggccgaacgcgtcggtcccga

SEQ ID NO: 100 - DNA, 60 bp, artificial
  primer
gttcggccgctgaaagcttcaggagaaaaaacccatgcaacagattgatccggccgcgtc

SEQ ID NO: 101 - DNA, 60 bp, artificial
  primer
tcgttttatttgatgcctctagagctcacatatgtcagccgcgcggcgcgcccatgaatc

SEQ ID NO: 102 - DNA, 60 bp, artificial
  primer
actgaccgaattcattaaagaggagaaaggtaccatgaccgacagacggatgctgatcgc

SEQ ID NO: 103 - DNA, 60 bp, artificial
  primer
gactgagcctttcgttttatttgatgcctctagatcatatcgggtaatgccgcggcgtgg

SEQ ID NO: 104 - DNA, 60 bp, artificial
  primer
accgaattcattaaagaggagaaaccacggtaccatgtcggcatcgaaacccaagctgcg

SEQ ID NO: 105 - DNA, 60 bp, artificial
  primer
ggctcgtattcatgggttttttctcctgggatcctcagtgcgaatgccgcggcaccgcgg

SEQ ID NO: 106 - DNA, 60 bp, artificial
  primer
gcattcgcactgaggatcccaggagaaaaaacccatgaatacgagccgttcgccgcgcta

SEQ ID NO: 107 - DNA, 60 bp, artificial
  primer
ttcgatgattccatagttaatttctcctaagctttcagcgctgaaacgggtcggccgcga

SEQ ID NO: 108 - DNA, 60 bp, artificial
  primer
cgtttcagcgctgaaagcttaggagaaattaactatggaatcatcgaatcggccggcgcg

SEQ ID NO: 109 - DNA, 60 bp, artificial
  primer
gttttatttgatgcctctagagctcagccatatgtcacgcgttgcgcgcgagcgcgaacc

SEQ ID NO: 110 - DNA, 60 bp, artificial
  primer
actgaccgaattcattaaagaggagaaaggtaccatgaacgggcccacgggcgaactcct

SEQ ID NO: 111 - DNA, 60 bp, artificial
  primer
gactgagcctttcgttttatttgatgcctctagatcacgttcgcgcacccgcgctcgcct

(19) United States
(12) Patent Application Publication
(10) Pub. No.: US 2017/0137855 A1
(43) Pub. Date: May 18, 2017

(54) RECOMBINANT CELLS AND METHODS FOR NONPHOSPHORYLATIVE METABOLISM

(71) Applicant: REGENTS OF THE UNIVERSITY OF MINNESOTA, Minneapolis, MN (US)

(72) Inventors: Kechun Zhang, Roseville, MN (US); Yi-shu Tai, Richmond, CA (US); Pooja Jambunathan, Minneapolis, MN (US)

(21) Appl. No.: 15/353,188

(22) Filed: Nov. 16, 2016

Related U.S. Application Data
(60) Provisional application No. 62/255,856, filed on Nov. 16, 2015.

(51) Int. Cl. C12P 19/02; C12P 7/40; C12N 9/88; C12N 9/04; C12N 9/18

(52) U.S. Cl. CPC C12P 19/02; C12N 9/0006; C12N 9/18; C12N 9/88; C12Y 402/01043; C12P 7/40; C12Y 101/01046; C12Y 301/01015; C12Y 402/01025

(57) ABSTRACT

This disclosure describes methods for screening heterologous coding regions (e.g., a single gene coding region, and operon, or other gene cluster) for the ability to genetically modify a host cell to perform nonphosphorylative biosynthesis of 2,5-dioxopentanoate and downstream derivatives thereof (e.g., 1,4-butanediol, glutamate, or glutaconate. This disclosure further describes recombinant cells modified to increase nonphosphorylative biosynthesis of 2,5-dioxopentanoate compared to an appropriate control cell (e.g., a wild-type cell or an otherwise genetically-modified cell) and methods of using making and using such recombinant cells.

### CROSS-REFERENCE TO RELATED APPLICATION

This application claims priority to U.S. Provisional Patent Application No. 62/255,856, filed Nov. 16, 2015, which is incorporated herein by reference.

### SEQUENCE LISTING

This application contains a Sequence Listing electronically submitted via EFS-Web to the United States Patent and Trademark Office as an ASCII text file entitled “11005220101_SequenceListing_ST25.txt” having a size of 19 kilobytes and created on Nov. 15, 2016. The information contained in the Sequence Listing is incorporated by reference herein.

### SUMMARY

This disclosure describes methods for screening heterologous coding regions (e.g., a single gene coding region, and operon, or other gene cluster) for the ability to genetically modify a host cell to perform nonphosphorylative biosynthesis of 2,5-dioxopentanoate and downstream derivatives thereof (e.g., 1,4-butanediol, glutamate, or glutaconate. This disclosure further describes recombinant cells modified to increase nonphosphorylative biosynthesis of 2,5-dioxopentanoate compared to an appropriate control cell (e.g., a wild-type cell or an otherwise genetically-modified cell) and methods of using making and using such recombinant cells.

Thus, in one aspect, this disclosure describes a recombinant cell modified to exhibit increased biosynthesis of 2,5-dioxopentanoate compared to a wild-type control. In some cases, the recombinant cell can include a genetic modification that inhibits TCA cycle production of 2-ketoglutarate. In some of these embodiments, the genetic modification can decrease oxidation of isocitrate to 2-ketoglutarate compared to a wild-type control.

Such a cell can further include one or more heterologous polynucleotides that encode an enzyme that performs a step of nonphosphorylative biosynthesis of 2,5-dioxopentanoate. Such as cell may be useful for screening the ability of enzyme(s) encoded by the heterologous polynucleotide to allow the recombinant cell to perform nonphosphorylative biosynthesis of 2,5-dioxopentanoate.

Thus, in another aspect, this disclosure describes a method of screening a heterologous polynucleotide for conferring to a host cell that ability to perform nonphosphorylative biosynthesis of 2,5-dioxopentanoate. In one embodiment, the method includes providing any embodiment of the recombinant cell summarized above, introducing into the cell at least one heterologous polynucleotide that encodes an enzyme that catalyzes a nonphosphorylative metabolic step of biosynthesis of D-xylose and growing the cell in culture medium that comprises D-xylose. In another embodiment, the method includes providing any embodiment of the recombinant cell summarized above, introducing into the cell at least one heterologous polynucleotide that encodes an enzyme that catalyzes a nonphosphorylative metabolic step of biosynthesis of L-arabinose, and growing the cell in culture medium that comprises L-arabinose.

In another aspect, this disclosure describes a recombinant cell that includes at least one heterologous polynucleotide that encodes an enzyme in a nonphosphorylative biosynthetic pathway that converts D-xylose to 2,5-dioxopentanoate. In some embodiments, the enzyme can include D-xylose dehydrogenase (XDH), D-xylonolactonase (XL), D-xylonate dehydratase (XD), or 2-keto-3-deoxy-D-xylonate dehydratase (KdxD). In some embodiments, the heterologous polynucleotide can include a polynucleotide from a Burkholderia spp. such as, for example, B. xenovorans polynucleotide. In one particular embodiment, the recombinant cell can include one or more of B. xenovorans polynucleotides DR64_8447, DR64_8448, DR64_8449, DR64_8450, or DR64_8452.

In another aspect, this disclosure describes a recombinant cell that includes at least one heterologous polynucleotide that encodes an enzyme in a nonphosphorylative biosynthetic pathway that converts L-arabinose to 2,5-dioxopentanoate. In some embodiments, the enzyme comprises L-arabinose dehydrogenase (ADH), L-arabinolactonase (AL), L-arabonate dehydratase (AD), and 2-keto-3-deoxy-L-arabonate dehydratase (KdaD). In some embodiments, the heterologous polynucleotide comprises a polynucleotide from a Burkholderia spp. such as, for example, a B. xenovorans polynucleotide, a B. ambifaria polynucleotide, or a B. thailandensis polynucleotide. In one particular embodiment, the recombinant cell can include one or more of B. ambifaria polynucleotides Bamb_4925, Bamb_4924, Bamb_4923, Bamb_4922, Bamb_4921, Bamb_4920, Bamb_4919, or Bamb_4915. In another particular embodiment, the recombinant cell can include one or more of B. thailandensis polynucleotides BTH_II 1632, BTH_II 1631, BTH_II 1630, BTH_II 1629, BTH_II 1628, BTH_II 1627, BTH_II 1626, or BTH_II 1625.

In yet another aspect, this disclosure describes a method for nonphosphorylatively biosynthesizing 2,5-dioxopentanoate. Generally, the method includes culturing a recombinant cell summarized immediately above under conditions effective for the recombinant cell to exhibit increased biosynthesis of 2,5-dioxopentanoate compared to a wild-type control. In some embodiments, the method includes culturing the cell in culture medium that provides D-xylose as a carbon source. In some embodiments, the method includes culturing the cell in culture medium that provides L-arabinose as a carbon source. In some embodiments, the method can further include converting the 2,5-dioxopentanoate to glutamate, 1,4-butanediol, or glutaconate.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Assimilation pathways of lignocellulosic sugars through nonphosphorylative metabolism. The pathway for D-xylose metabolism consists of D-xylose dehydrogenase (XDH), D-xylonolactonase (XL), D-xylonate dehydratase (XD), and 2-keto-3-deoxy-D-xylonate dehydratase (KdxD). The L-arabinose assimilation pathway is comprised of L-arabinose dehydrogenase (ADH), L-arabinolactonase (AL), L-arabonate dehydratase (AD), and 2-keto-3-deoxy-L-arabonate dehydratase (KdaD). The pathway for D-galacturonate metabolism was designed by using uronate dehydrogenase (UDH), D-galactarate dehydratase (GD), and 5-keto-4-deoxy-D-glucarate dehydratase (KdgD). The DOP produced from these feedstocks is then converted into 2-KG by 2-ketoglutarate semialdehyde dehydrogenase (KGSADH) which is a key intermediate of the TCA cycle.

FIG. 2. The growth platform to test functional nonphosphorylative gene clusters in E. coli and kinetic parameters of relevant enzymes. (A) The growth assay was designed based on the supply of TCA cycle intermediate, 2-ketoglutarate (2-KG). Isocitrate dehydrogenase gene (icd) was knocked out to cut off 2-KG production through glycolysis/TCA cycle. The nonphosphorylative pathway plasmids (pBDO-1 and pBDO-2 for D-xylose pathway, pBDO-3 for L-arabinose pathway, and pBDO-4 (a synthetic operon) and pBDO-2 for D-galacturonate) were then transformed into cells to compensate the production of 2-KG. (B) Strains BDO03 (BW25113 ΔxylA ΔyjhH ΔyagE), BDO04 (BW25113 ΔxylA ΔyjhH ΔyagE Δicd), and BDO04 transformed with plasmids pBDO-1 and pBDO-2 were grown in M9 minimum media supplemented with 5 g/l glucose and 5 g/l D-xylose. (C) Strains BDO05 (BW25113), BDO06 (BW25113 Δicd), and BDO06 transformed with plasmid pBDO-3 were grown in M9 minimum media supplemented with 5 g/l glucose and 5 g/l L-arabinose. (D) Strains BDO07 (BW25113 ΔuxaC ΔgarL), BDO08 (BW25113 ΔuxaC ΔgarL Δicd), and BDO08 transformed with plasmids pBDO-4 and pBDO-2 were grown in M9 minimum media supplemented with 5 g/l glucose and 5 g/l D-galacturonate. (E) Kinetic parameters of enzymes involved in non-phosphorylative pathways. All error bars are shown in (B), (C), and (D) and represent one SD (n=3).

FIG. 3. BDO production using different combinations of 2-ketoacid decarboxylase (KDC) and alcohol dehydrogenase (ADH) and different Kivd mutants. (A) BDO production pathway from DOP catalyzed by KDC and ADH. (B) Shake-flask production of BDO from D-xylose. Three KDC (Kivd, IpdC, and BFD) and five ADH (YqhD, AdhA, Adh6, YahK, and Yjgb) were examined. (C) Shake-flask production of BDO from L-arabinose and D-galacturonate using Kivd+YqhD. There was no BTO production using these substrates. (D) Shake-flask fermentation of BDO production from D-xylose. Ten Kivd mutants, S286Y, S286L, S286F, V461I, V461L, V461M, I465F, I465H, I465L, and I465M, were tested for BDO production. (E) The binding pocket of Kivd (PDB ID: 2VBG). Residues 5286, V461, and I465 were mutated to larger residues to improve the substrate specificity. (F) Kinetic parameters of WT Kivd and Kivd (V461I) for both DOP (the substrate for BDO production) and 2-keto-3-deoxy-D-xylonate (the substrate for BTO production). Error bars in (B), (C) and (D) represent one SD (n=3).

FIG. 4. Production of BDO from D-xylose, L-arabinose, and D-galacturonate in 1.3-L bioreactors. (A) Production of BDO from D-xylose. (B) Co-production of BDO and mevalonate from D-xylose and glucose. (C) Production of BDO from L-arabinose. (D) Production of BDO from D-galacturonate. Abbreviations: BDO, 1,4-butanendiol; D-Xyl, D-xylose; D-Gal, D-galacturonate; L-Ara, L-arabinose; MEV, mevalonate. Bioreactor experiments were performed at least triplicates for each substrate and results for one representative experiment are shown.

FIG. 5. Growth platform to mine putative nonphosphorylative clusters in E. coli and BDO production using these novel operons. (A) Strains BDO03 (BW25113 ΔxylA ΔyjhH ΔyagE), BDO04 (BW25113 ΔxylA ΔyjhH ΔyagE Δicd), BDO04 transformed with C. crescentus operon (pBDO-1 and pBDO-2) and BDO04 transformed with B. xenovorans operon (pBDO-23 and pBDO-24) were grown in M9 minimum media supplemented with 5 g/l glucose and 5 g/l D-xylose. (B) BDO production using newly identified B. xenovorans D-xylose operon and previous C. crescentus operon with Kivd and YqhD as downstream enzymes. (C) Strains BDO05 (BW25113), BDO06 (BW25113 Δicd), BDO06 transformed with B. multivorans operon (pBDO-3), BDO06 transformed with B. ambifaria operon (pBDO-25 and pBDO-26) and BDO06 transformed with B. thailandensis operon (pBDO-27 and pBDO-28) were grown in M9 minimum media supplemented with 5 g/l glucose and 5 g/l L-arabinose. (D) BDO production using previously identified B. thailandensis and novel B. multivorans and B. ambifaria L-arabinose operons with Kivd and YqhD as downstream enzymes. All error bars are shown in (B) and (D) and represent one SD (n=3).

FIG. 6. HPLC peak of in vitro BDO production using purified enzymes. Reaction was carried out in 100 mM Tris-HCl buffer (pH˜7.5) with 5 mM MgSO4 using 5 mM L-arabonate, 1 μM purified L-arabonate dehydratase (AraC), 1 μM purified L-KDA dehydratase (AraD), 1 μM purified Kivd, 1 μM purified YqhD, 5 mM thiamine diphosphate (ThDP) and 1 mM NADPH as co-factor. This reaction mixture was allowed to stand at room temperature for 30 mins and HPLC was performed on the resulting mixture. HPLC results showed that 2.5 mM L-arabonate was consumed and 1.7 mM BDO was produced after half hour. This validates the in vitro functionality of our proposed BDO pathway.

FIG. 7. Butanedial oxidation by endogenous E. coli enzymes. In vitro enzyme assay was performed using cell extract. The reaction was carried out in 100 mM Tris-HCl buffer (pH˜7.5) with 5 mM MgSO4 containing 1 μM L-arabonate dehydratase (AraC), 1 μM L-KDA dehydratase (AraD), 1 μM Kivd with 5 mM thiamine diphosphate (ThDP) and 1 mM NAD+ as co-factor. The reaction was initiated by adding 1 mM L-arabonate and 0.1 mg/ml of cell lysate and the absorbance was immediately measured at 340 nm using a spectrophotometer. A steady rise in absorbance was observed at 340 nm indicating oxidation of butanedial using NAD⁺ by endogenous E. coli enzymes. A reaction mixture containing 1 mM L-arabonate, 0.1 mg/ml cell lysate and 1 mM NAD⁺ without purified AraC, AraD and Kivd was used as negative control. This sample did not show any increase in absorbance at 340 nm.

FIG. 8. LC-MS data for 2-keto-3-deoxy-D-xylonate.

FIG. 9. LC-MS data for 2-keto-3-deoxy-L-arabonate.

FIG. 10. LC-MS data for 5-keto-4-deoxy-D-glucarate.

FIG. 11. LC-MS data for 2, 5-dioxopentanoate.

FIG. 12. Accumulation of 1,2,4-butanedtriol (BTO). (A) HPLC signal showing BTO accumulation with C. crescentus D-xylose operon. (B) Mechanism showing BTO formation due to promiscuous nature of 2-ketoacid decarboxylase (Kivd).

FIG. 13. Sequence identities of different D-xylose and L-arabinose operons. Sequence identities of B. ambifaria and B. thailandensis L-arabinose operons with respect to B. multivorans L-arabinose operon. Sequence identity of B. xenovorans D-xylose operon with respect to C. crescentus D-xylose operon. Enzymes are color coded—green: D-xylose/L-arabinose dehydrogenase; orange: D-xylonolactonase/L-arabinolactonase; blue: D-xylonate/L-arabonate dehydratase; purple: 2-keto-3-deoxy-D-xylonate/2-keto-3-deoxy-L-arabonate dehydratase; yellow: 2-ketoglutarate semialdehyde dehydrogenase.

### DETAILED DESCRIPTION OF ILLUSTRATIVE EMBODIMENTS

Conversion of lignocellulosic biomass into value-added products with high yields and rates provides significant environmental and economic benefits. This disclosure describes the engineering of an unconventional metabolism for the production of TCA cycle derivatives from D-xylose, L-arabinose, and D-galacturonate. This disclosure also describes a growth-based selection platform to identify several gene clusters functional in E. coli that can perform the nonphosphorylative assimilation of sugars into the TCA cycle in less than six steps. To demonstrate the application of this new metabolic platform, artificial biosynthetic pathways to 1,4-butanediol were built that exhibited a theoretical molar yield of 100%. By screening and engineering downstream pathway enzymes, 2-ketoacid decarboxylases, and alcohol dehydrogenases, E. coli strains were constructed that were capable of producing 12 g/l of BDO from D-xylose, 15.6 g/l of BDO from L-arabinose, and 16.5 g/l from D-galacturonate in 1.3 L bioreactors. The titers, rates, and yields are higher than those previously reported using conventional glycolysis and pentose phosphate pathways. This work demonstrates the potential of nonphosphorylative metabolism for biomanufacturing with improved biosynthetic efficiencies.

The use of edible biomass such as corn or sugarcane for biomanufacturing has affected food supply on a global scale and elevated food prices. In an effort to circumvent the contention of resources for “food versus chemical” purposes, lignocellulosic feedstock presents a promising solution. Lignocellulosic feedstock is the most abundant inedible biomass with an annual output of around 2×10¹¹ metric tons. Common sources of lignocellulose include corn stover, switchgrass, sugar beet pulp, and citrus peel. D-xylose, L-arabinose, and D-galacturonate make up more than one-third of the sugars in lignocellulose. Thus, efficient lignocellulosic fermentation processes involve the use of these materials.

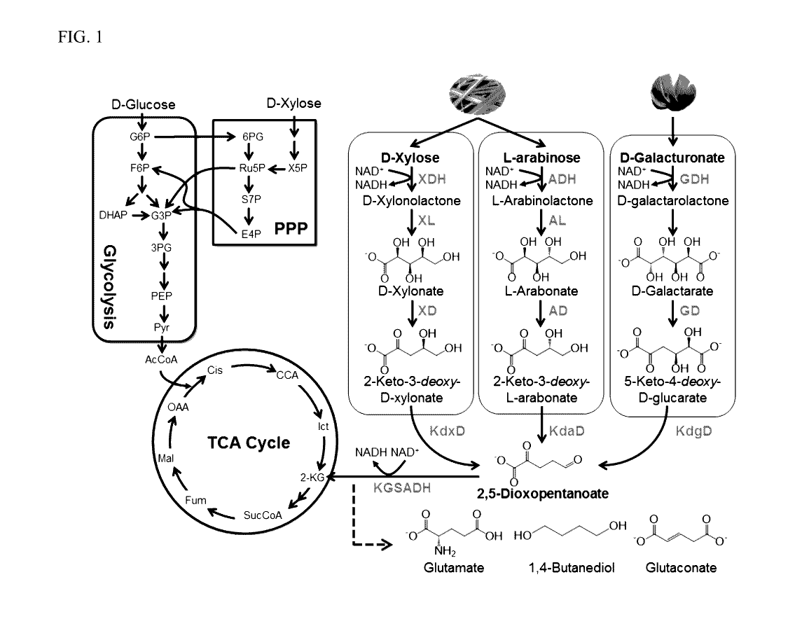

As the metabolic hub of the cell, the TCA cycle leads to a variety of high value bioproducts, including amino acids and industrial chemicals (FIG. 1). The conventional metabolic routes for carbon feedstocks to enter the TCA cycle are glycolysis and pentose phosphate pathways (PPP). These traditional metabolisms, however, involve lengthy reaction steps (>10 steps to TCA cycle) and complex regulations that limit the production yield and rate. For example, one reported pathway to produce 1,4-butanediol (BDO) requires 21 reaction steps, imposing significant difficulty on feasible metabolic engineering (Yim et al., 2011. Nat. Chem. Biol. 7:445-452). Moreover, after several decades of industrial practices, the fermentation yields of amino acids are still much lower than their theoretical maxima.

This disclosure describes an alternative, engineered unconventional metabolism that converts lignocellulosic materials directly into 2-ketoglutarate (2-KG) in less than six steps (FIG. 1). In this metabolic system, D-xylose is first converted into D-xylonolactone by D-xylose dehydrogenase (XDH), followed by hydrolysis to D-xylonate by D-xylonolactonase (XL). D-xylonate is subsequently dehydrated to 2-keto-3-deoxy-D-xylonate by D-xylonate dehydratase (XD), which is then converted to 2,5-dioxopentanoate (DOP) by 2-keto-3-deoxy-D-xylonate dehydratase (KdxD).

Through a similar metabolism, L-arabinose can be converted to 2,5-dioxopentanoate by L-arabinose dehydrogenase (ADH), L-arabinolactonase (AL), L-arabonate dehydratase (AD), and 2-keto-3-deoxy-L-arabonate dehydratase (KdaD).

The intermediate, 2,5-dioxopentanoate (DOP), produced from D-xylose or L-arabinose can be further oxidized to 2-KG, an intermediate of the TCA cycle, by 2-ketoglutarate semialdehyde dehydrogenase (KGSADH).

A comparable metabolism for the assimilation of uronic acids, such as D-galacturonate, has also been identified. Uronate dehydrogenase (UDH) can catalyze the transformation of D-galacturonate into D-galactaro-1,4-lactone; the lactone ring is then hydrolyzed either spontaneously or with the aid of a lactonase to form D-galactarate. D-Galactarate can be converted to 5-keto-4-deoxy-D-glucarate by D-galactarate dehydratase (GD) and then to 2,5-dioxopentanoate (DOP) by 5-keto-4-deoxy-D-glucarate dehydratase (KdgD). The DOP produced can again be further oxidized to 2-KG using a KGSADH. This alternative metabolism does not involve any phosphorylating reactions, making it more energy-efficient than the conventional pathways such as glycolysis and PPP. This nonphosphorylative pathway can be used as a shortcut to the TCA cycle, potentially accelerating the production of TCA cycle derivatives. In addition, the theoretical yield of 2-KG from these pentoses and uronic acids is 100 mol % through this metabolism, which is notably higher than that from pentose phosphate pathway (83 mol %).

Nonphosphorylative metabolism was discovered over fifty years ago. The full reconstitution of this pathway from sugars to 2-KG, however, has not been demonstrated in E. coli. A nonphosphorylative pathway from D-xylose has been reported (Liu et al., 2015, Metab. Eng. 29:135-141; Liu et al., 2013. Appl. Microbiol. Biotechnol. 97:3409-3417; Meijnen et al., 2009. Appl. Environ. Microbiol. 75:2784-2791; Radek et al., 2014. J. Biotechnol. 192:156-160), but the use of L-arabinose and/or D-galacturonate for chemical synthesis via this pathway has not been explored.

This disclosure describes a selection platform to discover nonphosphorylative gene clusters that are functional in E. coli. The platform was first tested using a previously identified gene cluster from C. crescentus, and then subsequently used for gene mining to assemble novel, putative gene clusters from various microorganisms that allow the nonphosphorylative assimilation of D-xylose, L-arabinose, and D-galacturonate. The corresponding enzymes were purified and their kinetic parameters were determined to validate their in vivo activities. The establishment of this alternative metabolism in E. coli provides a novel metabolic platform for biosynthesis of a variety of chemicals such as succinate, glutaconate, and the “glutamate family” of amino acids. Furthermore, it enables biotransformation of pharmaceutically important natural products catalyzed by 2-ketoglutarate-dependent dioxygenases.

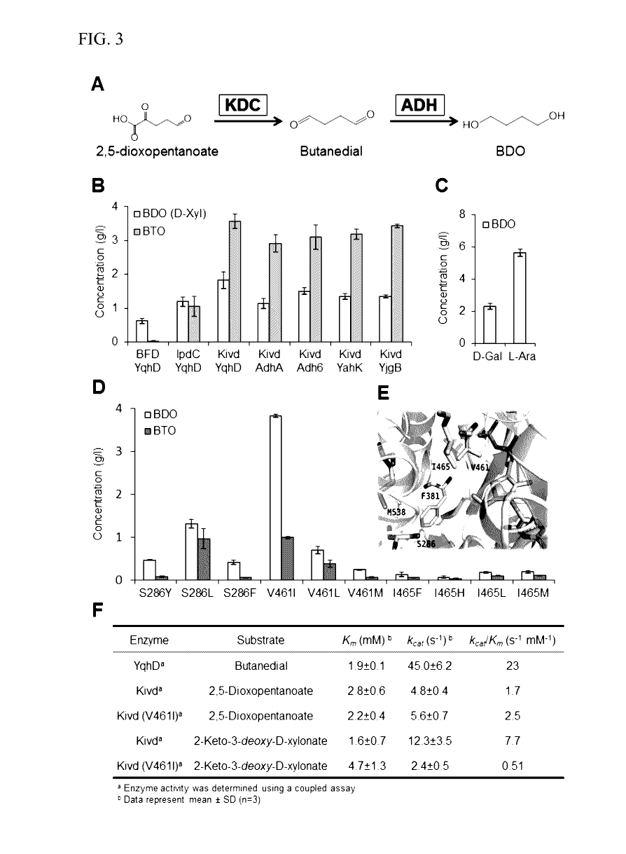

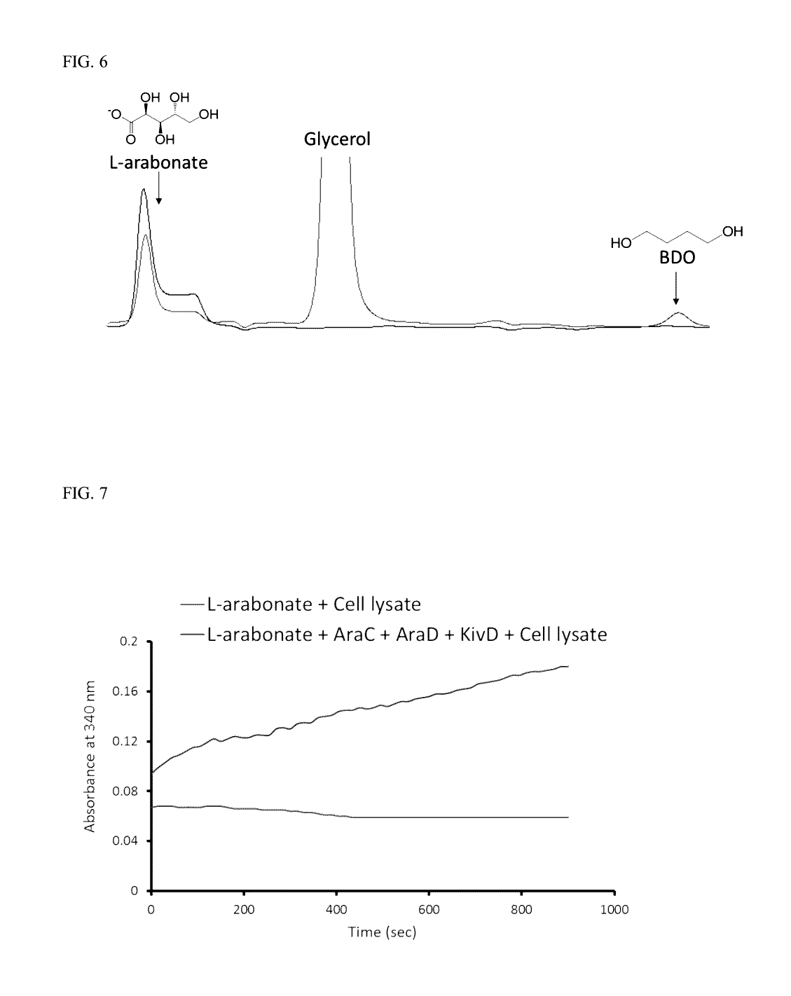

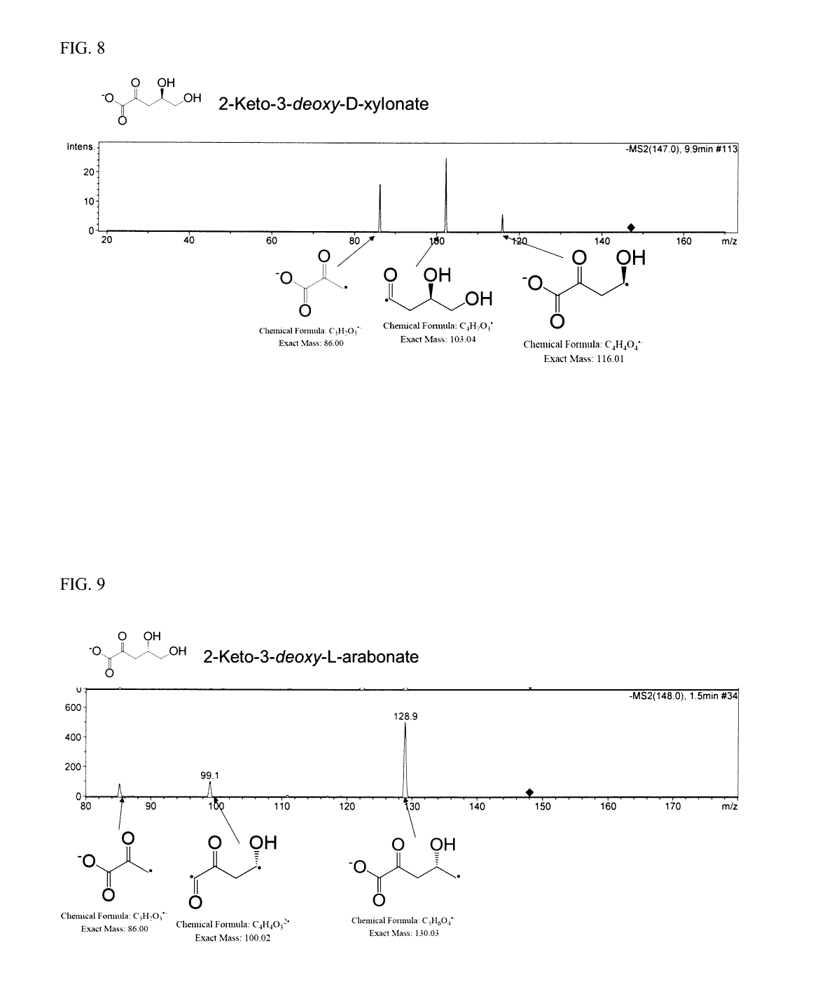

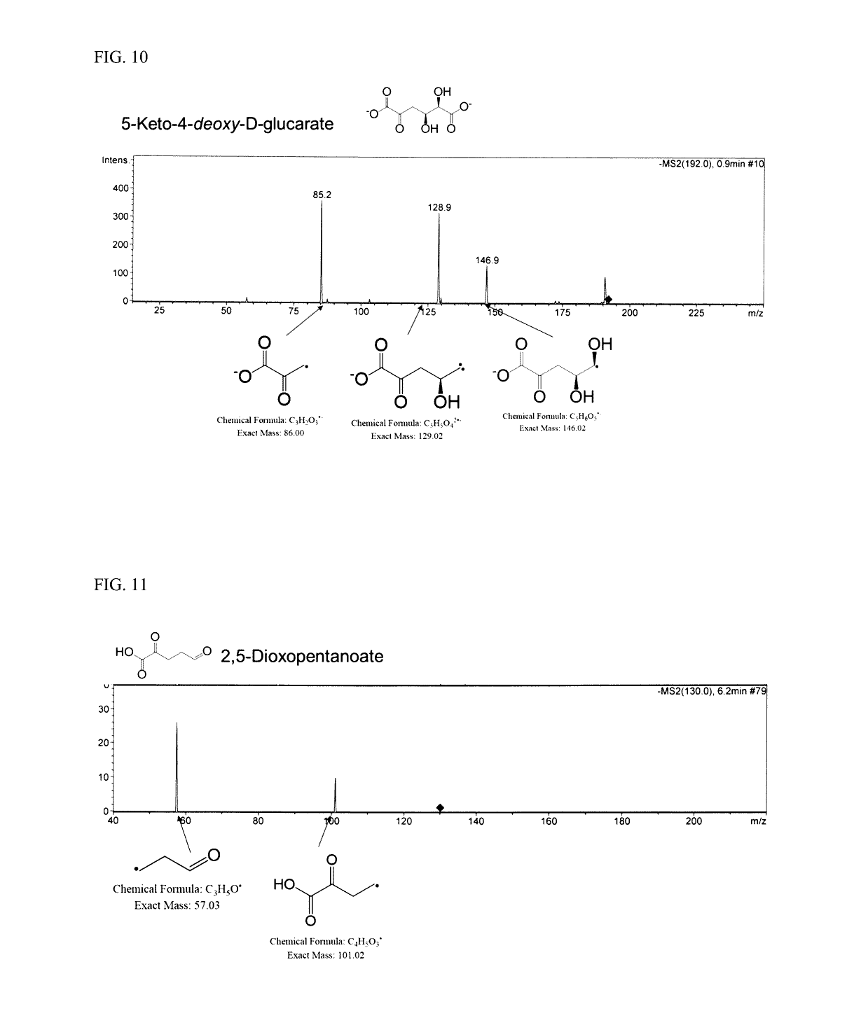

In one exemplary model embodiment, the engineered metabolism of D-xylose was used to supply 2-ketoglutarate for the biosynthesis of 1,4-butanediol (BDO), a raw material for many commercial products. This pathway uses a 2-ketoacid decarboxylase (KDC) to convert 2,5-dioxopentanoate into butanedial. Butanedial is then transformed into BDO by an alcohol dehydrogenase (ADH). The total biosynthetic pathway starting from the pentoses to BDO requires only six steps, which is less than one-third of the previously reported pathway (Yim et al., 2011. Nat. Chem. Biol. 7:445-452). Additionally, protein engineering techniques were used to reduce the accumulation of the byproduct, 1,2,4-butanetriol, by improving the selectivity of KDC towards 2,5-dioxopentanoate. The engineered KDC improved BDO titer from 1.83 g/l to 3.8 g/l at a yield of 63% of the theoretical maximum. The efficacy of the pathway described herein was validated by extensive enzymatic assays (FIG. 3F) and in vitro production experiments (FIG. 6). In addition, the production titer and yield (3.88 g/l BDO with a yield of 0.37 g/g D-xylose) indicates that the pathway described herein has a higher in vivo efficiency than other reported pathways.

Based on this nonphosphorylative platform, the sugar repertoire of the pathway was expanded to include L-arabinose and D-galacturonate. This disclosure describes producing 5.6 g/l BDO from L-arabinose and 2.3 g/l BDO from D-galacturonate, which has not been reported before. The scale-up feasibility for each substrate was then examined in 1.3 L bioreactors, where engineered strains were able to produce 12 g/l of BDO from D-xylose in 30 hours, 15.6 g/l BDO from L-arabinose in 75 hours, and 16.5 g/l of BDO from D-galacturonate in 90 hours.

Establishing the Nonphosphorylative Metabolism in E. coli

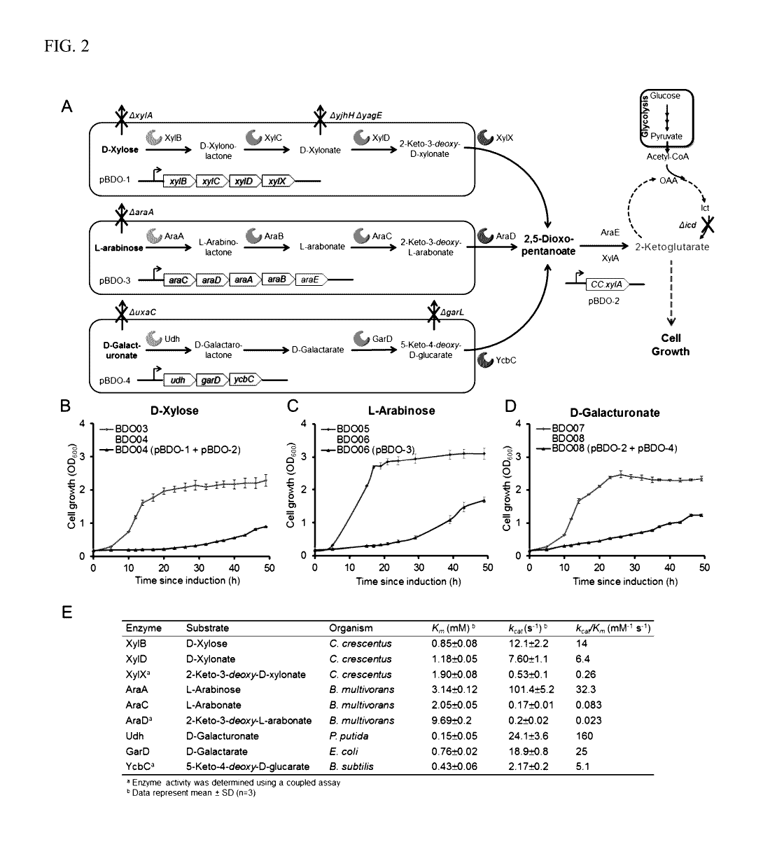

The selection platform based on cell growth illustrated in FIG. 2A was used to facilitate the discovery and engineering of nonphosphorylative gene clusters. Here, E. coli isocitrate dehydrogenase gene, icd, was knocked out so the oxidation of isocitrate to 2-KG was interrupted. Thus, the cells require an exogenous supply of 2-KG to grow. Since the alternate nonphosphorylative pathway can convert pentoses (such as D-xylose and L-arabinose) and uronic acids (such as D-galacturonate) to 2-KG, the activity of the pathway is coupled to cell growth. This platform can thus be used to screen for active gene clusters in E. coli. Alternatively, gene clusters can be further optimized by using directed evolution on the introduced pathway and identifying cells with improved growth.

To build the selection platform, the gene cluster xylBCDX (FIG. 2A) from C. crescentus was cloned into plasmid pBDO-1 (Table 2) where xylB encodes the XDH; xylC encodes the XL; xylD encodes XD and xylX encodes the KdxD (FIG. 1). The xylA coding region of the C. crescentus xylose operon, annotated as KGSADH, was cloned into a separate plasmid pBDO-2 (Table 2). Furthermore, to maximize flux of D-xylose through the nonphosphorylative pathway, the endogenous D-xylose (xylA) and D-xylonate (yagE, yjhH) consuming pathways in E. coli were knocked out, generating strain BDO03 (Table 2). Also, the icd coding region was deleted to generate strain BDO04, a 2-KG auxotroph that is incapable of producing 2-KG (Table 2). While strain BDO03 showed exponential growth in minimal media containing glucose and D-xylose, strain BDO04 showed almost no growth due to the disruption of the TCA cycle. BDO04 was then transformed with plasmids pBDO-1 and pBDO-2, and the resulting strain showed growth on mixed sugars with OD reaching approximately 1.0 after 50 hours (FIG. 2B). This could be attributed to the 2-KG produced from D-xylose via the nonphosphorylative pathway (pBDO-1 and pBDO-2).

Similar gene clusters encoding the L-arabinose assimilation pathway were also identified in several species of soil bacteria including Burkholderia spp., Pseudomonas saccharophilia, and Rhizobium spp. An L-arabinose assimilation gene cluster from Burkholderia multivorans was identified using BLAST, based on the previously identified L-arabinose gene cluster of Burkholderia thailandensis. To demonstrate the utility of the platform, the newly-identified L-arabinose gene cluster from Burkholderia multivorans araCDABE (BmulJ 5323-5321-5320-5316-5314), responsible for L-arabinose degradation to 2-KG, was cloned into plasmid pBDO-3 (Table 2). The araA coding region encodes the ADH; araB codes the AL; araC codes AD; araD was annotated as the KdaD, and araE encodes the KGSADH (FIG. 1). To eliminate the L-arabinose consumption pathways in E. coli, the endogenous E. coli araA coding region was knocked out to generate strain BDO05, which served as the positive control for the growth assay (Table 2). Strain BDO05 showed exponential growth in media containing both glucose and L-arabinose, but when icd gene was knocked out (BDO06), cells could not grow in the same media due to the disruption of the TCA cycle. When BDO06 strain was transformed with plasmid pBDO-3 containing the L-arabinose assimilation gene cluster, growth on glucose and L-arabinose media was restored due to the supplementation of 2-KG through the nonphosphorylative pathway. The BDO06 strain transformed with pBDO-3 grew to an OD of approximately 1.7 in 50 hours (FIG. 2C), thus establishing the in vivo activity of the novel B. multivorans L-arabinose gene cluster in E. coli.

Gene clusters with an analogous function for the hexuronic acid degradation were found in Bacillus species (Hosoya et al., 2002. FEMS Microbiol. Lett. 210:193-199) and Pseudomonas putida (Yoon et al., 2009. J. Bacteriol. 191:1565-73). To establish D-galacturonate pathway, a synthetic operon was designed that included the following coding regions: udh from Pseudomonas putida encoding the GDH, garD from E. coli encoding the GD, and ycbC from Bacillus subtilis encoding the putative KdgD (FIG. 1). This operon was cloned into plasmid pBDO-4 (Table 2). The plasmid pBDO-2 with the xylA coding region of C. crescentus was used to convert DOP to 2-KG. In order to maximize the flux of galacturonate via the heterologous pathway, coding regions encoding the pathways involved in consumption of either the substrate or intermediates were knocked out (ΔuxaC ΔgarL) resulting in BDO07 (Table 2). BDO07 grew exponentially after induction in the media containing both glucose and D-galacturonate. Similar to the D-xylose and L-arabinose pathways, when icd was knocked out (strain BDO08), cells could not grow since the strain is a 2-KG auxotroph. When strain BDO08 was transformed with plasmids pBDO-2 and pBDO-4, 2-KG was produced from D-galacturonate using the nonphosphorylative pathway, thus allowing cells to grow to an OD of approximately 1.25 in 50 hours (FIG. 2D).

### Validation of the Enzymatic Activities by In Vitro Assays

After demonstrating that the cloned metabolic pathways could function in vivo using a growth-based selection platform, the in vitro activities of the nonphosphorylative pathways were characterized. All the kinetic parameters are shown in FIG. 2E. In the D-xylose pathway, D-xylose dehydrogenase (XylB) and xylonate dehydratase (XylD) have k_{cat} values of 12.1 s⁻¹ and 7.6 s⁻¹, respectively. However, the enzyme 2-keto-3-deoxy-D-xylonate dehydratase (XylX) has a relatively low k_{cat} of 0.53 s⁻¹. Since XylX is also the enzyme with the highest Kₘ (1.9 mM) in the pathway, its specific constant (k_{cat}/Kₘ) is therefore the lowest (0.26 s⁻¹mM⁻¹) among the three enzymes. This indicates that XylX is the bottleneck enzyme in the D-xylose degradation pathway.

In the L-arabinose pathway, the first enzyme, L-arabinose dehydrogenase (AraA), has the highest k_{cat} value of 101.4 s⁻¹ while the downstream enzymes, L-arabonate dehydratase (AraC) and 2-keto-3-deoxy-L-arabonate dehydratase (AraD) have relatively low k_{cat} values of 0.17 s⁻¹ and 0.23 s⁻¹, respectively. The enzyme AraD has the highest Kₘ (9.7 mM) and a low k_{cat}, making it the rate-limiting step of the L-arabinose pathway.

For D-galacturonate pathway, uronate dehydrogenase (Udh) has the highest k_{cat} (24.1 s⁻¹) and lowest Kₘ (0.15 mM) among the three enzymes. Galactarate dehydratase (GarD) from E. coli has the highest Kₘ (0.76 mM) in the pathway but a much higher k_{cat} (18.9 s⁻¹) than 5-keto-4-deoxy-D-glucarate dehydratase (YcbC). Similar to D-xylose and L-arabinose pathway, the last enzyme (YcbC) in galacturonate pathway that produces DOP has the lowest specific constant and is considered the bottleneck enzyme in D-galacturonate degradation. These bottleneck enzymes from the three substrates could explain why transformants harboring these nonphosphorylative pathways did not grow as well as the wild type cells.

Identification of Enzymes to Convert DOP into BDO

With the establishment of the nonphosphorylative metabolism, its biosynthetic applications were explored by designing new synthetic pathways to BDO. 2,5-Dioxopentanoate (DOP) can be converted to BDO by a 2-ketoacid decarboxylase (KDC) and an alcohol dehydrogenase (ADH) (FIG. 3A). The BDO-producing pathways were designed using the following steps: (1) introducing the nonphosphorylative metabolism to convert the pentoses and hexuronic acid into a pool of DOP, (2) screening for the KDC variant that best converts DOP to butanedial, and (3) screening for the ADH variant that best reduces butanedial into BDO.

Since the D-xylose pathway has been partially established in E. coli, it was selected for enzyme screening. First, to convert DOP to butanedial, three KDCs were screened: 2-ketoacid decarboxylase (Kivd) from Lactococcus lactis (de la Plaza et al., 2004. FEMS Microbiol. Lett. 238:367-374), indolepyruvate decarboxylase (IpdC) from Salmonella typhimurium (Xiong et al., 2012. Sci. Rep. 2:311), and benzoylformate decarboxylase (BFD) from Pseudomonas putida (Iding et al., 2000. Chem. Eur. J. 6:1483-1495). The L. lactis Kivd was cloned into pBDO-5 S. typhimurium IpdC was cloned into pBDO-6, and P. putida BFD was cloned into pBDO-7 (Table 2). These plasmids also carried a promiscuous alcohol dehydrogenase YqhD from E. coli, for screening based on BDO production. To maximize the carbon flux towards the desired pathway, strain BDO03 was transformed with these plasmids individually along with the DOP-producing plasmid, pBDO-1. None of the strains used for BDO production have icd inactivation, and can thus utilize glucose for growth.

In the shake flask experiment, 20 g/l of D-xylose was used and the strains carrying Kivd, IpdC, and BFD produced 1.83 g/l, 1.20 g/l, and 0.63 g/l BDO, along with 3.56 g/l, 1.06 g/l, and 0.03 g/l 1,2,4-butanetriol (BTO), respectively, from D-xylose (FIG. 3B). Each of these enzymes is promiscuous enough to catalyze the decarboxylation of DOP to BDO. Of the three 2-ketoacid decarboxylases analyzed, Kivd best converted 2,5-Dioxopentanoate to butanedial.

Besides KDC, ADHs from different organisms were also investigated to see which combination would produce maximal titer of BDO. Other than YqhD, the following ADHs were screened: Lactococcus lactis alcohol dehydrogenase AdhA (Bastian et al., 2011. Metab. Eng. 13:345-352), Saccharomyces cerevisiae alcohol dehydrogenase Adh6 (Larroy et al., 2002. Biochem. J. 361:163-172), E. coli aldehyde reductase YahK (Jeudy et al., 2004. Protein Data Bank), and E. coli putative alcohol dehydrogenase YgjB (Oshima et al., 2006. Microbiol. 152:2129-2135). These enzymes were individually cloned after Kivd to build an expression cassette on a high copy plasmid as pBDO-8, pBDO-9, pBDO-10, and pBDO-11, respectively (Table 2). Strains carrying AdhA, Adh6, YahK and YgjB, could produce 1.15 g/l, 1.51 g/l, 1.36 g/l, and 1.36 g/l BDO together with 3.11 g/l, 2.92 g/l, 3.19 g/l, and 3.43 g/l BTO, respectively (FIG. 3B). Overall, the best combination was Kivd with YqhD allowing a yield of 0.15 g/g, which is only 25% of theoretical maximum. However, the byproduct BTO yield from D-xylose was 0.28 g/g, which is around two times higher than BDO. The apparent Kₘ and k_{cat} of Kivd towards DOP is 4.8 mM and 4.8 s⁻¹, respectively, and the apparent kinetic parameters of YqhD towards butanedial is 1.9 mM (Kₘ) and 45.0 s⁻¹ (k_{cat}) (FIG. 3F).

Based on the screening results of the D-xylose pathway, Kivd and YqhD (pBDO-5) were then applied to the L-arabinose and D-galacturonate pathways. For L-arabinose, the putative B. multivorans cluster araCDAB (BmulI 5323-5321-5320-5316) that can convert L-arabinose to DOP was cloned into plasmid pBDO-12. The strain BDO05 transformed with plasmids pBDO-12 and pBDO-5, was able to produce 5.65 g/l of BDO from 20 g/l L-arabinose in production experiments. For D-galacturonate, strain BDO07 was transformed with plasmids pBDO-4 and pBDO-5 and the engineered strain was capable of producing 2.34 g/l of BDO from 20 g/l D-galacturonate (FIG. 3C).

### Improving 1,4-Butanediol Production Pathway by Protein Engineering

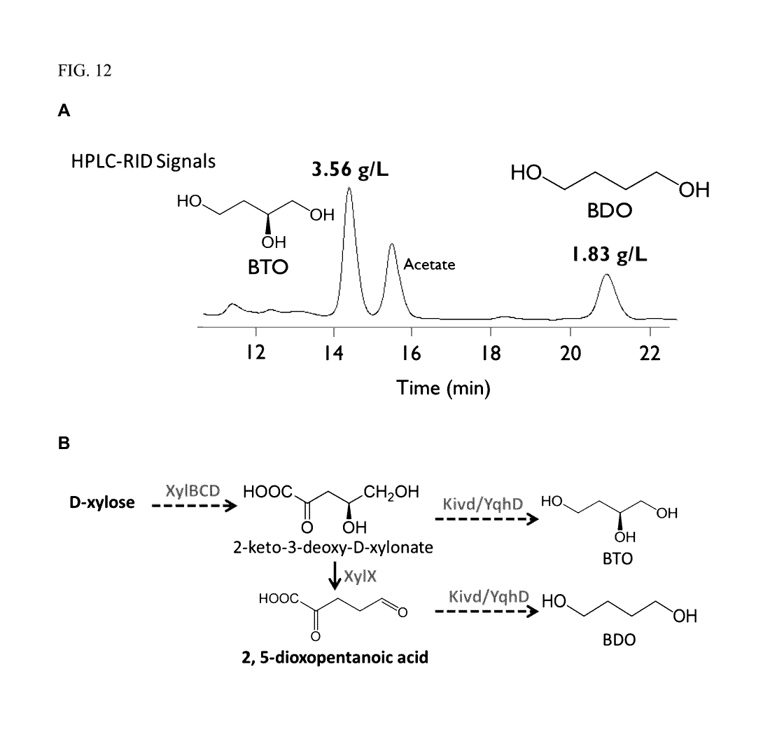

While the discovery of Kivd and YqhD allowed for the production of BDO, the promiscuous nature of Kivd did not provide good selectivity for the decarboxylation step in D-xylose pathway. BTO is produced by decarboxylation of the D-xylose intermediate, 2-keto-3-deoxy-D-xylonate (FIG. 12). This suggests that compared to DOP, Kivd prefers to bind to the intermediate 2-keto-3-deoxy-D-xylonate as a substrate, leading to a much higher titer of BTO than the target product, BDO. Conversely, there was no accumulation of BTO using L-arabinose as the substrate, indicating that Kivd is not active on the stereoisomer, 2-keto-3-deoxy-L-arabonate. To increase the production of BDO, protein engineering was used to improve Kivd selectivity towards DOP. According to the crystal structure (PDB ID: 2VBG), amino acid residues 5286, V461, and I465, in combination with the cofactor thiamine diphosphate (ThDP), delineate the active site of Kivd (FIG. 3E). Since 2-keto-3-deoxy-D-xylonate, with its extra hydroxyl group, is a bulkier substrate than DOP, the size of the binding site of Kivd was reduced to enhance its selectivity towards the smaller substrate. Ten mutants of Kivd, including S286Y, S286L, S286F, V461I, V461L, V461M, I465F, I465H, I465L and I465M, were constructed and tested.

The fermentation results indicated that the V461I mutant produced 3.83 g/l BDO with only 0.99 g/l BTO, which represents a yield of 0.37 g/g D-xylose which is 63% of the theoretical maximum. Compared with wild type Kivd, the BDO production titer with V461I mutant increased over two-fold. This can be attributed to the extra methyl group in isoleucine compared to valine, which reduces the size of the Kivd binding pocket, making it more selective towards DOP. Enzymatic assays also showed that the V461I Kivd mutant notably reduces the specific constant (k_{cat}/Kₘ) towards BTO substrate, 2-keto-3-deoxy-D-xylonate, from 7.7 to 0.5 mM⁻¹s⁻¹ (FIG. 3F), while improving the activity towards BDO substrate (DOP) from 1.7 to 2.5 mM⁻¹s⁻¹. Therefore, the enzyme characterization data is consistent with the fermentation results (FIG. 3D).

### Fermentation Scale-Up

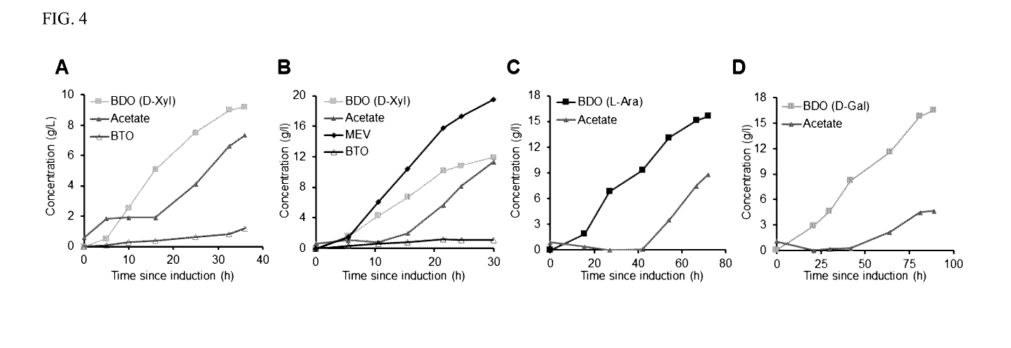

The scale-up feasibility of these BDO biosynthetic pathways was tested by fed-batch fermentation in 1.3-L bioreactors. For the D-xylose pathway, the recombinant strain BDO03, transformed with plasmids pBDO-1 and pBDO-5, was used. A mixture of glucose and D-xylose was applied as substrates during the fermentation process. The engineered strain produced 9.21 g/l BDO in 36 hours and consumed 42.1 g/l of D-xylose (FIG. 4A). Glucose was fed to support cell growth. To further exploit glucose for the production of value-added chemicals, another plasmid, pMEV-1 (Xiong et al., 2014. Proc. Natl. Acad. Sci. U.S.A. 111:8357-8362), was introduced into the engineered strain for the co-production of BDO and mevalonate (MEV). MEV is an important intermediate in the production of the branched lactone, β-methyl-δ-valerolactone, which could be used as building blocks for high-performing biobased polymers. The BDO03 strain transformed with pBDO-1, pBDO-5, and pMEV-1 was used for the fed-batch fermentation. The engineered strain produced 12.0 g/l BDO by consuming 46 g/l of D-xylose in 30 hours after induction (FIG. 4B). Not only was glucose efficiently utilized (20.2 mol % of glucose was converted into MEV), but the yield of BDO from D-xylose was also improved from 36% to 43% of the theoretical maximum by introducing MEV production pathway. Acetate started to accumulate to a final concentration of 11 g/l when cells entered stationary phase and inhibited further production of both BDO and MEV.

The L-arabinose recombinant strain BDO05, transformed with pBDO-5 and pBDO-12, was used in the fed-batch fermentation with a mixture of glucose and L-arabinose as feed. The engineered strain produced 15.6 g/l BDO in 72 hours and consumed 70.5 g/l of L-arabinose which resulted in a yield of 37% of the theoretical maximum. The final acetate concentration was 8.9 g/l, inhibiting further production of BDO (FIG. 4C). Similarly, the D-galacturonate strain BDO07, transformed with pBDO-4 and pBDO-5, was tested in a 1.3-L bioreactor. A mixture of glucose and D-galacturonate was fed to the bioreactor and the engineered strain produced 16.5 g/L of BDO from 50.5 g/L of D-galacturonate (70% of the theoretical maximum) in 90 hours (FIG. 4D).

### Identification of Alternative Gene Clusters

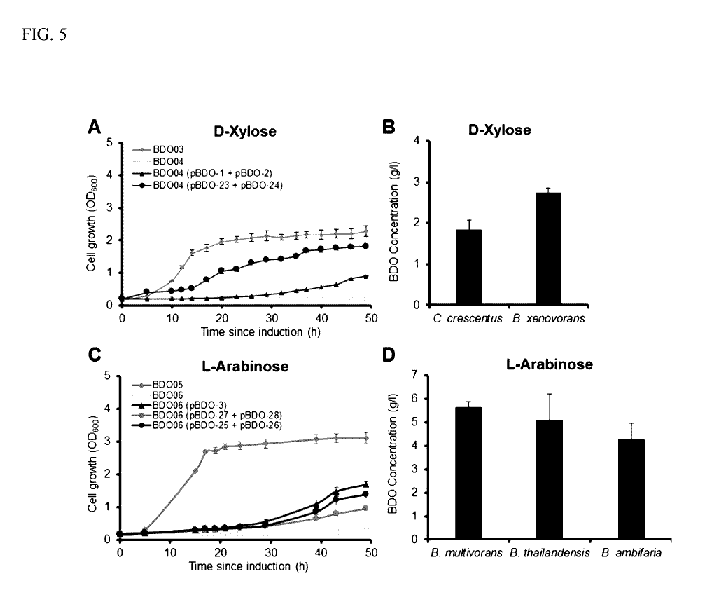

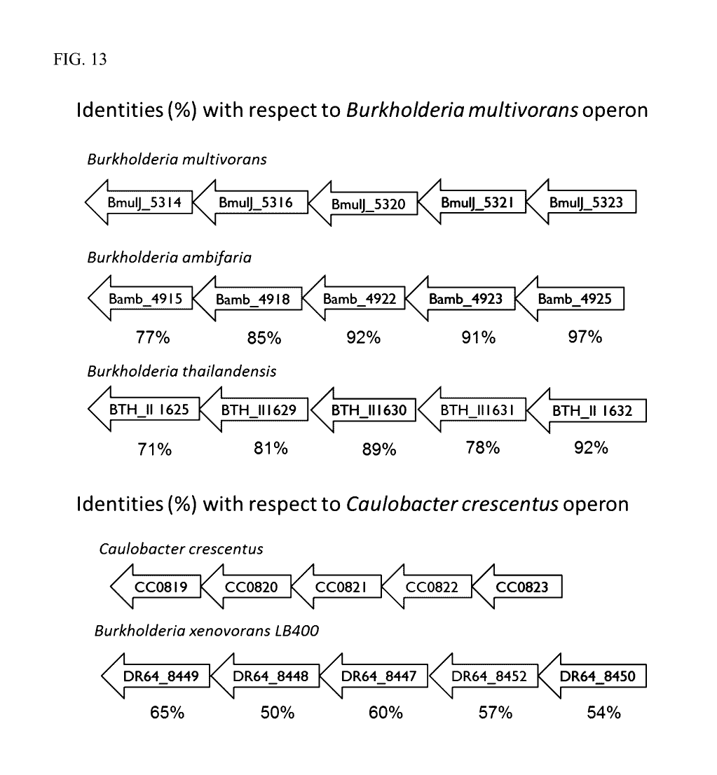

After successfully demonstrating the use of the nonphosphorylative metabolism of D-xylose, L-arabinose, and D-galacturonate for BDO production, alternative nonphosphorylative operons from other organisms were analyzed to identify alternatives that may show activity in E. coli. The growth-based selection platform that employs a 2-KG auxotroph was used to perform gene mining. Using BLAST, a putative operon from Burkholderia xenovorans LB400 (DR64_8447-DR64_8450, DR64_8452) for nonphosphorylative assimilation of D-xylose to 2-KG was identified. (FIG. 13). The coding regions for enzymes that convert D-xylose to DOP (DR64_8447-DR64_8450) were cloned into plasmid pBDO-23, and DR64_8452 was cloned into plasmid pBDO-24 to convert DOP to 2-KG. To test the in vivo activity of this gene cluster in E. coli, the 2-KG auxotroph BDO04 was transformed with plasmids pBDO-23 and pBDO-24. The B. xenovorans D-xylose gene cluster rescued the growth of E. coli with OD reaching ˜1.8 in 50 hours (FIG. 5A).

The production of BDO using the newly-identified B. xenovorans operon was evaluated. The recombinant strain, BDO03 transformed with plasmids pBDO-23 and pBDO-5, was able to produce 2.73 g/l BDO with no BTO accumulation (FIG. 5B). To further investigate this production profile, the in vitro enzyme activities of the new operon was evaluated.

TABLE 1
In vitro enzymatic activities of B. xenovorans and C. crescentus D-xylose operon

| | B. xenovorans | | | C. crescentus | | |
|---|---|---|---|---|---|---|
| | Kₘ | k_{cat} | k_{cat}/Kₘ | Kₘ | k_{cat} | k_{cat}/Kₘ |
| Enzyme name | (mM) | (s⁻¹) | (s⁻¹ mM⁻¹) | (mM) | (s⁻¹) | (s⁻¹ mM⁻¹) |
| D-xylose dehydrogenase | 1.97 ± 0.12 | 49.9 ± 4.2 | 25.3 | 0.85 ± 0.08 | 12.1 ± 2.2 | 14 |
| D-xylonate dehydratase | 1.52 ± 0.08 | 1.73 ± 0.3 | 1.14 | 1.18 ± 0.05 | 7.6 ± 1.1 | 6.4 |
| 2-keto-3-deoxy-D- | 8.96 ± 0.2 | 4.72 ± 0.5 | 0.53 | 1.9 ± 0.08 | 0.53 ± 0.1 | 0.26 |
| xylonate dehydratase^{a} | | | | | | |

^{a}Enzyme activity was determined using a coupled assay

The 2-keto-3-deoxy-D-xylonate dehydratase of B. xenovorans (DR64_8450) has a 9-fold higher k_{cat} (4.7 s⁻¹) compared to the corresponding dehydratase (XylX) of C. crescentus (0.53 s⁻¹). This can explain why there was no BTO accumulation using B. xenovorans operon. Thus, the selection strategy could be used to discover highly active enzymes from different microorganisms. These enzymes could be combinatorially assembled into synthetic operons for potential biosynthesis.

For L-arabinose, operons from two other Burkholderia species were tested: an uncharacterized, putative Burkholderia ambifaria gene cluster (Bamb_4925-4918, Bamb_4915) and a previously identified, uncharacterized Burkholderia thailandensis gene cluster (BTH_II1632-1625; Brouns et al., 2006. J. Biol. Chem. 281:27378-27388), both of which had high sequence similarity (FIG. 13) to B. multivorans L-arabinose operon. The putative B. ambifaria coding regions Bamb_4925-Bamb_4918, which convert L-arabinose to DOP, were cloned into plasmid pBDO-25, and the coding region Bamb_4915, that converts DOP to 2-KG, was cloned into plasmid pBDO-26. Similarly, the B. thailandensis coding regions responsible for DOP production (BTH_II1625, BTH_II1629, BTH_II1630 and BTH_II1632) were cloned into plasmid pBDO-27, and BTH_II1631, that converts DOP to 2-KG, was cloned into plasmid pBDO-28. Both gene clusters rescued the growth of the 2-KG auxotroph, BDO06, to an OD of ˜1.5 in 50 hours (FIG. 5C) via the nonphosphorylative pathway. After establishing the in vivo activities of both L-arabinose gene clusters, they were used for BDO production. The B. ambifaria strain, BDO03 with plasmids pBDO-25 and pBDO-5, produced 4.3 g/l BDO; the B. thailandensis strain, BDO03 with plasmids pBDO-27 and pBDO-5, produced 5 g/l BDO in production experiments (FIG. 5D). Similar to B. multivorans operon, both B. ambifaria and B. thailandensis gene clusters did not produce any BTO.

Nonphosphorylative metabolism allows assimilation of lignocellulosic sugars or sugar acids into the TCA cycle intermediate, 2-KG. This disclosure describes the complete nonphosphorylative metabolism of D-xylose, L-arabinose, and D-galacturonate to 2-KG in a model host organism, E. coli. A selection platform using a 2-KG auxotroph was used to discover coding regions that are functional in E. coli. In particular, the platform was applied to identify a new nonphosphorylative D-xylose operon from B. xenovorans that has a more active 2-keto-3-deoxy-D-xylonate dehydratase than the previously reported one from C. crescentus. The discovery of alternative enzymes and/or operons enables one to further engineer nonphosphorylative metabolic pathways. Establishing such pathways can serve as a new biosynthetic platform for TCA cycle derivatives which have extensive applications. In this disclosure, BDO production is used as a model for such engineered metabolism.

To establish the downstream pathway to BDO, several different decarboxylases and dehydrogenases were screened. The best enzyme combination for BDO production was the 2-ketoacid decarboxylase (Kivd) from L. lactis and the endogenous alcohol dehydrogenase (YqhD) of E. coli. Protein engineering on Kivd successfully improved BDO titer from D-xylose by more than 100% and reduced BTO accumulation. Thus, directed evolution can be combined with other engineering strategies to further improve the selectivity.

In addition, the C. crescentus D-xylose gene cluster, the B. multivorans L-arabinose operon, and the D-galacturonate synthetic operon were tested in a 1.3-L bioreactor to study the scale-up feasibility. Acetate accumulation and inefficient co-utilization of sugars caused by carbon catabolite repression are two limiting factors in the processes. The strain could thus be further improved by knocking out acetate-producing pathways and/or relieving carbon catabolite repression with the overexpression of D-xylose, L-arabinose, or D-galacturonate transporters. Fermentation process engineering or strain evolution can also be applied for optimization.

While the results reported in this disclosure demonstrate the production of an exemplary commodity chemical, 1,4-butanediol (BDO), the nonphosphorylative platform also can be extended to produce several TCA cycle derivatives including glutamate, glutaconate, and 1-butanol, among others. The growth selection platform provides an effective and robust tool to screen better enzymes and/or identify nonphosphorylative pathways for other substrates. Compared to conventional metabolic pathways such as glycolysis and PPP, the fewer-steps and higher theoretical yields involved in the use of nonphosphorylative pathways can make lignocellulosic bioproducts more economically feasible.

Thus, in one aspect, this disclosure describes recombinant microbes and methods of biosynthesizing a 2,5-dioxopentanoate. As used herein, “biosynthesizing,” “a biosynthesized” compound, and other terms having “biosynthesis” as a root refer to the characteristic of at least one step of the synthesis of the compound being performed by a microbe. While describes herein in the context of exemplary embodiments, illustrated in FIG. 1, there are multiple biosynthetic pathways by which 2,5-dioxopentanoate (and products downstream of 2,5-dioxopentanoate) may be biosynthesized.

One can distinguish a biosynthesized compound as described herein from a similar compound produced by conventional chemical processes from, for example, a petroleum-based material by the ratio of ¹⁴C to ¹²C in a sample of the compound. A sample of the compound that is biosynthesized will possess a measurable amount of ¹⁴C isotopes incorporated into the compound molecules, while a sample of the compound prepared from petroleum-based materials will possess negligible levels of ¹⁴C. Thus, a sample or composition that includes biosynthesized fumarate analog will possess a ¹⁴C/¹²C ratio greater than zero. In some cases, a sample or composition that includes a biosynthesized compound can have a ¹⁴C/¹²C ratio greater than 0.25×10⁻¹² such as, for example, a ¹⁴C/¹²C ratio from 0.25×10⁻¹² to 1.2×10⁻¹².

Biosynthesized 2,5-dioxopentanoate can be produced by a method, described in more detail below, in which a host cell is modified to be a recombinant cell that can exhibit increased biosynthesis of 2,5-dioxopentanoate compared to a wild-type control. In some cases, the wild-type control may be unable to produce 2,5-dioxopentanoate and, therefore, an increase in the biosynthesis of 2,5-dioxopentanoate may reflect any measurable biosynthesis of 2,5-dioxopentanoate. In certain embodiments, an increase in the biosynthesis of 2,5-dioxopentanoate can include biosynthesis sufficient for a culture of the microbial cell to accumulate 2,5-dioxopentanoate to a predetermine concentration.

In some embodiments, an increase in the biosynthesis of 2,5-dioxopentanoate can be reflected by the accumulation of a downstream product for which 2,5-dioxopentanoate is a biosynthetic intermediate. Exemplary downstream biosynthetic products includes, for example, glutamate, 1,4-butanediol (BD), and glutaconate. Thus, in some embodiments, an increase in the biosynthesis of 2,5-dioxopentanoate can include biosynthesis sufficient for a culture of the microbial cell to accumulate glutamate, 1,4-butanediol (BD), or glutaconate to a predetermine concentration.

In one exemplary embodiment, the predetermined concentration of 1,4-butanediol (BDO) may be any predetermined concentration of BDO suitable for a given application. Thus, a predetermined concentration may be, for example, a concentration of at least 0.1 g/L such as, for example, at least 0.25 g/L, at least 0.5 g/L, at least 1.0 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, at least 10 g/L, at least 20 g/L, at least 50 g/L or at least 100 g/L. In certain specific embodiments, the method can result in the accumulation of, for example, at about 12 g/L of BDO with D-xylose as a carbon source, about 16 g/L of BDO with L-arabinose as a carbon source, or about 17 g/L of BDO with D-galacturonate as a carbon source.

Thus, in another aspect, this disclosure describes methods of biosynthesizing 2,5-dioxopentanoate. The method generally includes culturing an appropriate recombinant cell—described in more detail below—under conditions effective for the recombinant cell to exhibit increased biosynthesis of 2,5-dioxopentanoate compared to a comparable wild-type control.

The recombinant cell modified to exhibit increased biosynthesis of 2,5-dioxopentanoate compared to a wild-type control can be any suitable recombinant cell. While exemplary embodiments described herein include a genetically modified E. coli host cell, the recombinant cells described herein can be constructed, and the methods of making and using the recombinant cells can be performed, using any suitable host cell. Thus, the recombinant cell can be, or be derived from, any suitable microbe including, for example, a prokaryotic microbe or a eukaryotic microbe. As used herein, the term “or derived from” in connection with a microbe simply allows for the “host cell” to possess one or more genetic modifications before being modified to exhibit increased biosynthesis of the fumarate analog. Thus, the term “recombinant cell” encompasses a “host cell” that may contain nucleic acid material from more than one species before being modified to exhibit an increase in biosynthesis of the fumarate analog.

In some embodiments, the host cell may be selected to possess one or more natural physiological activities. For example, the host cell may be photosynthetic (e.g., cyanobacteria) or may be cellulolytic (e.g., Clostridium cellulolyticum).

In some embodiments, the recombinant cell may be, or be derived from, a eukaryotic microbe such as, for example, a fungal cell. In some of these embodiments, the fungal cell may be, or be derived from, a member of the Saccharomycetaceae family such as, for example, Saccharomyces cerevisiae, Candida rugosa, or Candida albicans. In some of these embodiments, yield can be improved by at least partially knocking out activity of the host cell's native mesaconate consuming pathways.

In other embodiments, the recombinant cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis), members of the Clostridiaceae family (e.g., Clostridium cellulolyticum) and, for example, members of the Streptococcaceae family (e.g., Lactococcus lactis). In other cases, the bacterium may be a member of the phylum Cyanobacteria. Here again, in some of these embodiments, yield can be improved by at least partially knocking out activity of the host cell's native metabolic pathways that may consume the fumarate analog

In some embodiments, the recombinant cell can exhibit increased activity compared to a wild-type control of one or more enzymes involved in a metabolic biosynthetic pathway for producing 2,5-dioxopentanoate. As used herein, the terms “activity” with regard to particular enzyme refers to the ability of a polypeptide, regardless of its common name or native function, to catalyze the conversion of the enzyme's substrate to a product, regardless of whether the “activity” as less than, equal to, or greater than the native activity of the identified enzyme. Methods for measuring the biosynthetic activities of cells are routine and well known to those of ordinary skill in the art.

As used herein, an increase in catalytic activity can be quantitatively measured and described as a percentage of the catalytic activity of an appropriate wild-type control. The catalytic activity exhibited by a genetically-modified polypeptide can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild-type control.

Alternatively, an increase in catalytic activity may be expressed as at an increase in k_{cat} such as, for example, at least a two-fold increase, at least a three-fold increase, at least a four-fold increase, at least a five-fold increase, at least a six-fold increase, at least a seven-fold increase, at least an eight-fold increase, at least a nine-fold increase, at least a 10-fold increase, at least a 15-fold increase, or at least a 20-fold increase in the k_{cat} value of the enzymatic conversion.

An increase in catalytic activity also may be expressed in terms of a decrease in Kₘ such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the Kₘ value of the enzymatic conversion.

A decrease in catalytic activity can be quantitatively measured and described as a percentage of the catalytic activity of an appropriate wild-type control. The catalytic activity exhibited by a genetically-modified polypeptide can be, for example, no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, no more than 1% of the activity, or 0% of the activity of a suitable wild-type control.

Alternatively, a decrease in catalytic activity can be expressed as an appropriate change in a catalytic constant. For example, a decrease in catalytic activity may be expressed as at a decrease in k_{cat} such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the k_{cat} value of the enzymatic conversion.

A decrease in catalytic activity also may be expressed in terms of an increase in Kₘ such as, for example, an increase in Kₘ of at least two-fold, at least three-fold, at least four-fold, at least five-fold, at least six-fold, at least seven-fold, at least an eight-fold, at least nine-fold, at least 10-fold, at least 15-fold, at least 20-fold, at least 25-fold, at least 30-fold, at least 35-fold, at least 40-fold, at least 45-fold, at least 50-fold, at least 75-fold, at least 100-fold, at least 150-fold, at least 200-fold, at least 230-fold, at least 250-fold, at least 300-fold, at least 350-fold, or at least 400-fold.

In some embodiments, the recombinant cells may be designed to grow in cell culture that includes culture medium that has, as a carbon source, D-xylose, L-arabinose, and/or D-galacturonate. Thus, the recombinant cell can include at least one enzyme that provides increased enzymatic activity in a biosynthetic pathway that converts D-xylose, L-arabinose, and/or D-galacturonate to 2,5-dioxopentanoate.

In some embodiments, the recombinant cell can exhibit increased activity of at least one enzyme involved in a biosynthetic pathway that converts D-xylose to 2,5-dioxopentanoate. Exemplary enzymes that may be included in such a pathway include, for example, a D-xylose dehydrogenase (XDH), a D-xylonolactonase (XL), a D-xylonate dehydratase (XD), and/or a 2-keto-3-deoxy-D-xylonate dehydratase (KdxD). In some embodiments, one or more of the enzymes may be, or be derived from, an enzyme natively expressed by a member of the Caulobacteraceae family such as, for example, Caulobacter crescentus.

In some embodiments, the recombinant cell can exhibit increased activity of at least one enzyme involved in a biosynthetic pathway that converts L-arabinose to 2,5-dioxopentanoate. Exemplary enzymes that may be included in such a pathway include, for example, an L-arabinose dehydrogenase (ADH), an L-arabinolactonase (AL), an L-arabonate dehydratase (AD), and/or a 2-keto-3-deoxy-L-arabonate dehydratase (KdaD). In some embodiments, one or more of the enzymes may be, or be derived from, an enzyme natively expressed by a member of the Burkholderiaceae family such as, for example, a Burkholderia spp. (e.g., B. multivorans); a member of the Pseudomonadaceae family such as, for example, a Pseudomonas spp. (e.g., P. saccarophilia); and/or a member of the Rhizobiaceae family such as, for example, a Rhizobium spp.

In some embodiments, the recombinant cell can exhibit increased activity of at least one enzyme involved in a biosynthetic pathway that converts D-galacturonate to 2,5-dioxopentanoate. Exemplary enzymes that may be included in such a pathway include, for example, a uronate dehydrogenase (UDH), a D-galactarate dehydratase (GD), and/or 5-keto-4-deoxy-D-glucarate dehydratase (KdgD). In some embodiments, one or more of the enzymes may be, or be derived from, an enzyme natively expressed by a member of the Bacillaceae family such as, for example, a Bacillus spp. (e.g., B. subtilis) or a member of the Pseudomonadaceae family such as, for example, a Pseudomonas spp. (e.g., P. putida).

In some embodiments, the recombinant cell can exhibit increased activity of at least one enzyme involved in a biosynthetic pathway that converts 2,5-dioxopentanoate to 2-ketoglutarate (2-KG), glutamate, 1,4-butanediol, or glutaconate. Exemplary enzymes that may be included in such a pathway include, for example, a 2-ketoglutarate semialdehyde dehydrogenase (KGSADH), a 2-ketoacid decarboxylase (KDC) and/or an alcohol dehydrogenase (ADH). Exemplary KGSADHs include, for example, XylA from C. crescentus. Exemplary KDCs include, for example, 2-ketoacid decarboxylase (Kivd) from Lactococcus lactis, indolepyruvate decarboxylase (IpdC) from Salmonella typhimurium, and benzoylformate decarboxylase (BFD) from Pseudomonas putida.

Exemplary ADHs include, for example, YqhD from E. coli, AdhA from Lactococcus lactis, Adh6 from Saccharomyces cerevisiae, YahK from E. coli, and YgjB from E. coli. In one particular embodiment, the recombinant cell can include 2-ketoacid decarboxylase (Kivd) from Lactococcus lactis and YqhD from E. coli to covert 2,5-dioxopentanoate to 1,4-butanediol.

In some cases, an enzyme may be engineered to improve the efficiency of its enzymatic activity. This can include, for example, increasing the specificity of the enzyme for a particular substrate. As shown in FIG. 3E, one can model the active site of an enzyme and design mutants that include an amino acid substitution at a residue within the active site. FIG. 3B shows the results of an exemplary enzyme re-design of Kivd in which 10 different single amino acid substitutions were generated and tested. Thus, for example, the biosynthetic pathway that converts 2,5-dioxopentanoate to 1,4-butanediol can include a genetically-modified Kivd that includes an amino acid substitutions at position 286, position 461, or position 465. In one particular embodiment, the modified Kivd includes a substitution of an isoleucine for the valine at position 461.

In some cases, the recombinant cell can exhibit a decrease in the activity of at least one endogenous metabolic pathway that is competitive with the engineered pathway. As used herein, “endogenous” refers to a polynucleotide, an enzyme, an activity, etc. that originates from the host cell. Such a reduction in activity may be referred to herein as a “knockout” of the stated activity. A “knockout” can exhibit any degree of reduced activity compared to a wild-type and does not necessary connote a complete elimination of the stated enzyme activity. A “knockout” can therefore result from, for example, deleting at least a part of the coding region of the enzyme whose activity is reduced.

For example, any embodiment of the recombinant cell described above can exhibit reduced activity of isocitrate dehydrogenase (ICD), which normally converts isocitrate to 2-ketoglutarate in the TCA cycle. Such a knockout causes the cell to obtain 2-ketoglutarate from a source other than the TCA cycle such as, for example, the oxidation of 2,5-dioxopentanoate. As another example, embodiments that involve using D-xylose as a carbon source to produce 2,5-dioxopentanoate can exhibit reduced activity of at least one enzyme in metabolic pathway for the consumption of D-xylose and/or D-xylonate (e.g., XylA, YagE, and/or YjhH in E. coli). As another example, embodiments using L-arabinose as a carbon source to produce 2,5-dioxopentanoate can exhibit reduced activity of at least one enzyme in metabolic pathway for the consumption of L-arabinose (e.g., AraA in E. coli). As yet another example, embodiments that use D-galacturonate to produce 2,5-dioxopentanoate can exhibit reduced activity of at least one enzyme in a metabolic pathway for the consumption of D. galaturonate (e.g., UxaC and/or GarL in E. coli).

In constructing the various recombinant cells described herein, a heterologous polynucleotide encoding a heterologous polypeptide may be inserted into a vector. As used herein, a vector is a replicating polynucleotide such as, for example, a plasmid, phage, or cosmid, to which another polynucleotide may be inserted so as to bring about the replication of the inserted polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can permit, for example, further cloning—i.e., a cloning vector—or expression of the polypeptide encoded by the coding region—i.e., an expression vector. The term vector includes, but is not limited to, plasmid vectors, viral vectors, cosmid vectors, or artificial chromosome vectors. In one embodiment, the vector is a plasmid. Selecting a vector can depend upon a variety of desired characteristics in the resulting construct, such as a selection marker, vector replication rate, and the like. In embodiments that include two or more heterologous polynucleotides, each of which encodes a heterologous polypeptide, two or more heterologous polynucleotides may be provided in a single vector or may be provide on separate vectors.

An expression vector optionally includes regulatory sequences operably linked to the coding region. The polynucleotides described herein are not limited by the use of any particular promoter, and a wide variety of promoters are known. Promoters act as regulatory signals that bind RNA polymerase in a cell to initiate transcription of a downstream (3′ direction) coding region. The promoter used can be a constitutive or an inducible promoter. It can be, but need not be, heterologous with respect to the host cell. Exemplary promoters include, for example, trp, tac, and T7. In embodiments in which two or more heterologous polynucleotides are provided on a single vector, they may be placed under the control of separate regulatory sequences or, alternatively, may be placed under the common control of a single regulatory sequence.

“Coding sequence” or “coding region” refers to a nucleotide sequence that encodes a polypeptide and, when placed under the control of appropriate regulatory sequences, expresses the encoded polypeptide. The boundaries of a coding region are generally determined by a translation start codon at its 5′ end and a translation stop codon at its 3′ end. As used herein, the term “polypeptide” refers broadly to a polymer of two or more amino acids joined together by peptide bonds. The term “polypeptide” also includes molecules that contain more than one polypeptide joined by disulfide bonds, ionic bonds, or hydrophobic interactions, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. The term “polypeptide” does not connote a specific length of a polymer of amino acids, nor does it imply or distinguish whether the polypeptide is produced using recombinant techniques, chemical or enzymatic synthesis, or is naturally occurring.

“Regulatory sequence” refers to a nucleotide sequence that regulates expression of a coding region to which it is operably linked. Nonlimiting examples of regulatory sequences include, for example, promoters, transcription initiation sites, translation start sites, translation stop sites, and terminators. “Operably linked” refers to a juxtaposition wherein the components are in a relationship permitting them to function in their intended manner. A regulatory sequence is “operably linked” to a coding region when it is joined in such a way that expression of the coding region is achieved under conditions compatible with the regulatory sequence.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Bacterial and Growth Conditions

The E. coli strains used in this study are listed in Table 2. XL10-Gold was used for cloning and BL21 was used for protein expression and purification. Most of the other strains were derived from the wild-type E. coli K-12 strain BW25113 (Datsenko et al., 2000. Proc. Natl. Acad. Sci. U.S.A. 97:6640-6645). P1 phages of xylA, yjhH, yagE, icd, uxaC, and garL were obtained from the Keio collection (Baba et al., 2006. Mol. Syst. Biol. 2:10.1038). The phages were used to transfect the corresponding strain for the construction of targeted knockout strains. All the knockout strains were then transformed with pCP20 plasmid to remove the kanamycin marker. The correct knockouts were verified by colony PCR. Unless otherwise stated, these E. coli strains were grown in test tubes at 37° C. in 2×YT rich medium (16 g/l Bacto-tryptone, 10 g/l yeast extract and 5 g/l NaCl) supplemented with appropriate antibiotics (ampicillin 100 mg/l and kanamycin 50 mg/l).

TABLE 2
E. coli strains and plasmids used.

| Name | Relevant genotype | Reference |
|---|---|---|
| Strains | | |
| BW25113 | rrnB_{T14} ΔlacZ_{WJ16}hsdR514 ΔaraBAD_{AH33} ΔrhaBAD_{LD78} | 1 |
| XL10- | Tet^{R}Δ (mcrA)183 Δ (mcrCB-hsaSMR-mrr)173 endA1supE44 | Stratagene |
| Gold | thi-1 recA1 | |
| BL21 | E. coli B F- dcmompThsdS(r_{B}− m_{B}−) gal [malB⁺]_{K-12}(λ^{S}) | 2 |
| BDO01 | BW25113 ΔxylA | This work |
| BDO02 | BW25113 ΔxylAΔyjhH | This work |
| BDO03 | BW25113 ΔxylAΔyjhHΔyagE | This work |
| BDO04 | BW25113 ΔxylΔyjhHΔyagEΔicd | This work |
| BDO05 | BW25113 | 1 |
| BDO06 | BW25113 Δicd | This work |
| BDO07 | BW25113 ΔgarLΔuxaC | This work |
| BDO08 | BW25113 ΔgarLΔuxaCΔicd | This work |
| Plasmids | | |
| pBDO-1 | P15A origin, Kan^{R}, P_{L}lacO₁: xylB-xylC-xylD-xylX | This work |
| pBDO-2 | ColE1 origin, Amp^{R}, P_{L}lacO₁: xylA(CC) | This work |
| pBDO-3 | P15A origin, Kan^{R}, P_{L}lacO₁: araC-araD-araA-araB-araE | This work |
| pBDO-4 | P15A origin, Kan^{R}, P_{L}lacO₁: udh-garD-ycbC | This work |
| pBDO-5 | ColE1 origin, Amp^{R}, P_{L}lacO₁: kivd -yqhD | This work |
| pBDO-6 | ColE1 origin, Amp^{R}, P_{L}lacO₁: ipdC -yqhD | This work |
| pBDO-7 | ColE1 origin, Amp^{R}, P_{L}lacO₁: BFD-yqhD | This work |
| pBDO-8 | ColE1 origin, Amp^{R}, P_{L}lacO₁: kivd-adhA | This work |
| pBDO-9 | ColE1 origin, Amp^{R}, P_{L}lacO₁: kivd-adh6 | This work |
| pBDO-10 | ColE1 origin, Amp^{R}, P_{L}lacO₁: kivd-yahK | This work |
| pBDO-11 | ColE1 origin, Amp^{R}, P_{L}lacO₁: kivd-yjgB | This work |
| pBDO-12 | P15A origin, Kan^{R}, P_{L}lacO₁: araC-araD-araA-araB | This work |
| pBDO-13 | ColE1 origin, Amp^{R}, P_{L}lacO₁: kivd(S286Y)-yqhD | This work |
| pBDO-14 | ColE1 origin, Amp^{R}, P_{L}lacO₁: kivd(S286L)-yqhD | This work |
| pBDO-15 | ColE1 origin, Amp^{R}, P_{L}lacO₁: kivd(S286F)-yqhD | This work |
| pBDO-16 | ColE1 origin, Amp^{R}, P_{L}lacO₁: kivd(V461I)-yqhD | This work |
| pBDO-17 | ColE1 origin, Amp^{R}, P_{L}lacO₁: kivd(V461L)-yqhD | This work |
| pBDO-18 | ColE1 origin, Amp^{R}, P_{L}lacO₁: kivd(V461M)-yqhD | This work |
| pBDO-19 | ColE1 origin, Amp^{R}, P_{L}lacO₁: kivd(I465F)-yqhD | This work |
| pBDO-20 | ColE1 origin, Amp^{R}, P_{L}lacO₁: kivd(I465H)-yqhD | This work |
| pBDO-21 | ColE1 origin, Amp^{R}, P_{L}lacO₁: kivd(I465L)-yqhD | This work |
| pBDO-22 | ColE1 origin, Amp^{R}, P_{L}lacO₁: kivd(I465M)-yqhD | This work |
| pBDO-23 | P15A origin, Kan^{R}, P_{L}lacO₁: DR64_8447-8448-8449-8450 | This work |
| pBDO-24 | ColE1 origin, Amp^{R}, P_{L}lacO₁: DR64_8452 | This work |
| pBDO-25 | P15A origin, Kan^{R}, P_{L}lacO₁: Bamb_4925-4923-4922-4918 | This work |
| pBDO-26 | ColE1 origin, Amp^{R}, P_{L}lacO₁: Bamb_4915 | This work |
| pBDO-27 | P15A origin, Kan^{R}, P_{L}lacO₁: BTH_II1632-1630-1629-1625 | This work |
| pBDO-28 | ColE1 origin, Amp^{R}, P_{L}lacO₁: BTH_II1631 | This work |
| pMEV-1 | pUC origin, Spec^{R}, P_{L}lacO₁: atoB-mvaS-mvaE | 3 |

1 (Datsenko et al., 2000. Proc. Natl. Acad. Sci. U.S.A. 97: 6640-6645)
2 (Studier et al., 1986. J. Mol. Biol. 186: 113-130)
3 (Bastian et al, 2011. Metab. Eng. 13: 345-352)

### Plasmid Construction

All the primers used in this study were ordered from Eurofins MWG Operon and are listed in Table 3. PCR reactions were carried out with PHUSION High-Fidelity DNA polymerase (New England Biolabs Inc., Ipswich, Mass.) according to the manufacturer's instructions. The sequences of all the plasmids produced were verified by restriction mapping and DNA sequencing.

TABLE 3
Primers used

| Primer Name | Sequence | |
|---|---|---|
| xylBAcc-F | GGGCCCGGTACCATGTCCTCAGCCATCTATCCCAGCCT | SEQ ID NO: 1 |
| xylBBamHI-R | GGGCCCGGATCCTTAACGCCAGCCGGCGTCGATCCAGT | SEQ ID NO: 2 |
| xylCBamHI-F | GGGCCCGGATCCAGGAGAAATTAACTATGACCGCTCAAGTC | SEQ ID NO: 3 |
| | ACTTGCGTATG | |
| xylCNhe-R | GGGCCCGCTAGCTTAGACAAGGCGGACCTCATGCTGGG | SEQ ID NO: 4 |
| xylDNheI-F | GGGCCCGCTAGCAGGAGAAATTAACTATGAGGTCCGCCTTG | SEQ ID NO: 5 |
| | TCTAACCGCAC | |
| xylDHind-R | GGGCCCAAGCTTTTAGTGGTTGTGGCGGGGCAGCTTGG | SEQ ID NO: 6 |
| xylXHind-F | GGGCCCAAGCTTAGGAGAAATTAACTATGGTTTGTCGGCGG | SEQ ID NO: 7 |
| | CTTCTAGCATG | |
| xylXBlpRem-R | GCGCAGCTGGCGTTGTTGTCCTTGGCCTTTCTGAGCAGCAG | SEQ ID NO: 8 |
| | GGCCGAACGACCTTCGAA | |
| xylXBlpRem-F | TTCGAAGGTCGTTCGGCCCTGCTGCTCAGAAAGGCCAAGGA | SEQ ID NO: 9 |
| | CAACAACGCCAGCTGCGC | |
| xylXBlpI-R | GGGCCCGCTCAGCTTAGAGGAGGCCGCGGCCGGCCAGGT | SEQ ID NO: 10 |
| CC0822Acc-F | GGGCCCGGTACCATGACCGACACCCTGCGCCATTACAT | SEQ ID NO: 11 |
| CC0822Xba-R | GGGCCCTCTAGATTACGACCACGAGTAGGAGGTTTTGG | SEQ ID NO: 12 |
| BFDAcc-F | GGGCCCGGTACCATGGCTTCGGTACACGGCACCACATA | SEQ ID NO: 13 |
| BFDSphI-R | GGGCCCGCATGCTTACTTCACCGGGCTTACGGTGCTTA | SEQ ID NO: 14 |
| KIVDAcc-F | GGGCCCGGTACCATGTATACAGTAGGAGATTACCTATT | SEQ ID NO: 15 |
| KIVDSphI-R | GGGCCCGCATGCTTATGATTTATTTTGTTCAGCAAATA | SEQ ID NO: 16 |
| IPDCAcc-F | GGGCCCGGTACCATGCAAAACCCCTATACCGTGGCCGA | SEQ ID NO: 17 |
| IPDCSphI-R | GGGCCCGCATGCTTATCCCCCGTTGCGGGCTTCCAGCG | SEQ ID NO: 18 |
| YqhDSphI-F | GGGCCCGCATGCAGGAGAAATTAACTATGAACAACTTTAAT | SEQ ID NO: 19 |
| | CTGCACACCCC | |
| YqhDXbaI-R | GGGCCCTCTAGATTAGCGGGCGGCTTCGTATATACGGC | SEQ ID NO: 20 |
| Adh6-SphI-F | GGGCCCGCATGCAGGAGATATACCATGTCTTATCCTGAGAA | SEQ ID NO: 21 |
| | ATTTGAAGG | |
| Adh6-XbaI-R | GGGCCCTCTAGACTAGTCTGAAAATTCTTTGTCGTAGC | SEQ ID NO: 22 |
| yahK-SphI-F | GGGCCCGCATGCAAGGAGATATACCATGAAGATCAAAGCTG | SEQ ID NO: 23 |
| | TTGGTGCATA | |
| yahK-XbaI-R | GGGCCCTCTAGATTAGTCTGTTAGTGTGCGATTATCGA | SEQ ID NO: 24 |
| yjgB-SphI-F | GGGCCCGCATGCAAGGAGATATACCATGTCGATGATAAAAA | SEQ ID NO: 25 |
| | GCTATGCCGC | |
| yjgB-XbaI-R | GGGCCCTCTAGATTAAAAATCGGCTTTCAACACCACGC | SEQ ID NO: 26 |
| adhA-SphI-F | GGGCCCGCATGCAAGGAGATATACCATGAAAGCAGCAGTAG | SEQ ID NO: 27 |
| | TAAGACACAA | |
| adhA-XbaI-R | GGGCCCTCTAGATTATTTAGTAAAATCAATGACCATTC | SEQ ID NO: 28 |
| pZAAcc-R | GGGCCCGGTCTCAATAGTTTCTCCTCTTTAATGAATTCGGT | SEQ ID NO: 29 |
| | CA | |
| KdaBS-F | GGGCCCGGTCTCACTATGGTACCATGAGCCGTATCAGAAAA | SEQ ID NO: 30 |
| | GCACCCGC | |
| KdaBS-R | GGGCCCGGTCTCATTACTTAAACCGTCGCGGCTTTTTTCGG | SEQ ID NO: 31 |
| | AA | |
| GarD-F | GGGCCCGGTCTCAGTAAGCTAGCAGGAGAAATTAACTATGG | SEQ ID NO: 32 |
| | CCAACATCGAAATCAGACA | |
| GarD-Acc-R | GGGCCCGGTCTCAACCGCCATCAGGCCGTACGGCGTACC | SEQ ID NO: 33 |
| GarD-Acc-F | GGGCCCGGTCTCACGGTGCCCGTCATTAAAATGGCAACCCG | SEQ ID NO: 34 |
| GarD-R | GGGCCCGGTCTCACAGGTTAGGTCACCGGTGCCGGGTTAAA | SEQ ID NO: 35 |
| | CA | |
| udh-F | GGGCCCGGTCTCACCTGAAGCTTAGGAGAAATTAACTATGA | SEQ ID NO: 36 |
| | CCACTACCCCCTTCAATCG | |
| udh-Bsa-R | GGGCCCGGTCTCATGTCTCGATGCCGTAGCGGTCAAAGTAG | SEQ ID NO: 37 |
| udh-Bsa-F | GGGCCCGGTCTCAGACAGTCAGCATTCGCATCGGCTCGTCG | SEQ ID NO: 38 |
| udh-R | GGGCCCGGTCTCAGCGGTTAGTTGAACGGGCCGGCCACGGC | SEQ ID NO: 39 |
| | GA | |
| pZA-F | GGGCCCGGTCTCACCGCGCTGAGCTCTAGAGGCATCAAATA | SEQ ID NO: 40 |
| | AAACGAAAG | |
| KivdS286Y-R | TTTAAATGATGAGTGAAGGCTCCTGTTGAGTAGTCTGTGAG | SEQ ID NO: 41 |
| | TTTAACTCCAAGCATCA | |
| KivdS286Y-F | TGATGCTTGGAGTTAAACTCACAGACTACTCAACAGGAGCC | SEQ ID NO: 42 |
| | TTCACTCATCATTTAAA | |
| KivdS286L-R | TAAATGATGAGTGAAGGCTCCTGTTGAGAGGTCTGTGAGTT | SEQ ID NO: 43 |
| | TAACTCCAAGCATCAGG | |
| KivdS286L-F | CCTGATGCTTGGAGTTAAACTCACAGACCTCTCAACAGGAG | SEQ ID NO: 44 |
| | CCTTCACTCATCATTTA | |
| KivdS286F-R | TTAAATGATGAGTGAAGGCTCCTGTTGAGAAGTCTGTGAGT | SEQ ID NO: 45 |
| | TTAACTCCAAGCATCAG | |
| KivdS286F-F | CTGATGCTTGGAGTTAAACTCACAGACTTCTCAACAGGAGC | SEQ ID NO: 46 |
| | CTTCACTCATCATTTAA | |
| KivdV461I-R | TTGATTTGGTCCATGAATTTCTCTTTCGATTGTATAACCAT | SEQ ID NO: 47 |
| | CATTATTGATAATAAAGC | |
| KivdV461I-F | GCTTTATTATCAATAATGATGGTTATACAATCGAAAGAGAA | SEQ ID NO: 48 |
| | ATTCATGGACCAAATCAA | |
| KivdV461L-R | TTGATTTGGTCCATGAATTTCTCTTTCGAGTGTATAACCAT | SEQ ID NO: 49 |
| | CATTATTGATAATAAAGC | |
| KivdV461L-F | GCTTTATTATCAATAATGATGGTTATACACTCGAAAGAGAA | SEQ ID NO: 50 |
| | ATTCATGGACCAAATCAA | |
| KivdV461M-R | TGATTTGGTCCATGAATTTCTCTTTCCATTGTATAACCATC | SEQ ID NO: 51 |
| | ATTATTGATAATAA | |
| KivdV461M-F | TTATTATCAATAATGATGGTTATACAATGGAAAGAGAAATT | SEQ ID NO: 52 |
| | CATGGACCAAATCA | |
| KivdI465F-R | TCATTGTAGCTTTGATTTGGTCCATGGAATTCTCTTTCGAC | SEQ ID NO: 53 |
| | TGTATAACCATCAT | |
| KivdI465F-F | ATGATGGTTATACAGTCGAAAGAGAATTCCATGGACCAAAT | SEQ ID NO: 54 |
| | CAAAGCTACAATGA | |
| KivdI465H-R | TCATTGTAGCTTTGATTTGGTCCATGGTGTTCTCTTTCGAC | SEQ ID NO: 55 |
| | TGTATAACCATCAT | |
| KivdI465H-F | ATGATGGTTATACAGTCGAAAGAGAACACCATGGACCAAAT | SEQ ID NO: 56 |
| | CAAAGCTACAATGA | |
| KivdI465L-R | TCATTGTAGCTTTGATTTGGTCCATGCAGTTCTCTTTCGAC | SEQ ID NO: 57 |
| | TGTATAACCATCAT | |
| KivdI465L-F | ATGATGGTTATACAGTCGAAAGAGAACTGCATGGACCAAAT | SEQ ID NO: 58 |
| | CAAAGCTACAATGA | |
| KivdI465M-R | TCATTGTAGCTTTGATTTGGTCCATGCATTTCTCTTTCGAC | SEQ ID NO: 59 |
| | TGTATAACCATCAT | |
| KivdI465M-F | ATGATGGTTATACAGTCGAAAGAGAAATGCATGGACCAAAT | SEQ ID NO: 60 |
| | CAAAGCTACAATGA | |
| Kivd-BamHI-R | GGGCCCGGATCCATGTATACAGTAGGAGATTACCTATT | SEQ ID NO: 61 |
| Kivd-Xba R | GGGCCCTCTAGATTATGATTTATTTTGTTCAGCAAATA | SEQ ID NO: 62 |
| YqhD-BamHI-R | GGGCCCGGATCCATGAACAACTTTAATCTGCACACCCC | SEQ ID NO: 63 |
| YqhD-XbaI-R | GGGCCCTCTAGATTAGCGGGCGGCTTCGTATATACGGC | SEQ ID NO: 64 |
| XvlA-BamHI-F | GGGCCCGGATCCATGACCGACACCCTGCGCCATTACAT | SEQ ID NO: 65 |
| XylA-XbaI-R | GGGCCCTCTAGATTACGACCACGAGTAGGAGGTTTTGG | SEQ ID NO: 66 |
| XvlX-BamHI-F | GGGCCCGGATCCATGGTTTGTCGGCGGCTTCTAGCATG | SEQ ID NO: 67 |
| XylX-XbaI-R | GGGCCCTCTAGATTAGAGGAGGCCGCGGCCGGCCAGGT | SEQ ID NO: 68 |
| AraC-Acc65I-F | CCGAATTCATTAAAGAGGAGAAAGGTACCATGTCGGCAACG | SEQ ID NO: 69 |
| | AAACCCAGGCTGCGCTCC | |
| AraC-NheI-R | GATCCTGCGTCAGTCAAACGGCGGGCTAGCTCAGTGCGAGT | SEQ ID NO: 70 |
| | GGCTCGGCACCTCCGCGCC | |
| AraD-NheI-F | GAGGTGCCGAGCCACTCGCACTGAGCTAGCCCGCCGTTTGA | SEQ ID NO: 71 |
| | CTGACGCAGGATCCGAACC | |
| AraD-remBlpI-R | GGCTCATCGTGCGCTCCTTGGTTCGTTGCTCACCGTGCCCA | SEQ ID NO: 72 |
| | GCGCAGCACGAGCGGATCG | |
| AraD-remBlpI-F | CGATCCGCTCGTGCTGCGCTGGGCACGGTGAGCAACGAACC | SEQ ID NO: 73 |
| | AAGGAGCGCACGATGAGCC | |
| AraA-HindIII-R | TCGATGCTCAGGCGGCGCGCACGCAAGCTTTCAGCGGCCGA | SEQ ID NO: 74 |
| | ACGCTTCGGTGTCGACGCG | |
| AraA-HindIII-F | GACACCGAAGCGTTCGGCCGCTGAAAGCTTGCGTGCGCGCC | SEQ ID NO: 75 |
| | GCCTGAGCATCGATTATCG | |
| AraB-NdeI-R | TTGCGCCGCGTCGCCGCCATATGTCAGGTTCCGACGCCGCG | SEQ ID NO: 76 |
| | CTTCAGTGCGAATCGCGCG | |
| AraB-NdeI-F | CTGAAGCGCGGCGTCGGAACCTGACATATGGCGGCGACGCG | SEQ ID NO: 77 |
| | GCGCAACCCGACCTGGGCC | |
| AraE-BlpI-R | TCGTTTTATTTGATGCCTCTAGAGCTCAGCTCAGATCGGGT | SEQ ID NO: 78 |
| | AATGCCGCGGCGCGGTCTG | |
| AraB-BlpI-R | CCAGGTCGGGTTGCGCCGCGTCGCCGCGCTCAGCTCAGGTT | SEQ ID NO: 79 |
| | CCGACGCCGCGCTTCAGTG | |
| DR64-8447-F | AATTCATTAAAGAGGAGAAAGGTACCATGTCGTACGCAATC | SEQ ID NO: 80 |
| | TATCCCAGCCT | |
| DR64-8447-R | ACAGGGGGATGAATTTTCATAGTTAATTTCTCCTGGATCCT | SEQ ID NO: 81 |
| | TATTCTCCGTACCACCCGG | |
| DR64-8448-F | CCGGGTGGTACGGAGAATAAGGATCCAGGAGAAATTAACTA | SEQ ID NO: 82 |
| | TGAAAATTCATCCCCCTGT | |
| DR64-8448-R | CGCGGTGTGGATGCTGACATAGTTAATTTCTCCTGCTAGCT | SEQ ID NO: 83 |
| | TATTGCGCGAAGCCCCATT | |
| DR64-8449-F | AATGGGGCTTCGCGCAATAAGCTAGCAGGAGAAATTAACTA | SEQ ID NO: 84 |
| | TGTCAGCATCCACACCGCG | |
| DR64-8449-R | GATGGAGAAGTTGCGGACATAGTTAATTTCTCCTAAGCTTT | SEQ ID NO: 85 |
| | TAGTGCGAATGCCTCGGAT | |
| DR64-8450-F | ATCCGAGGCATTCGCACTAAAAGCTTAGGAGAAATTAACTA | SEQ ID NO: 86 |
| | TGTCCGCAACTTCTCCATC | |
| DR64-8450-R | CGTTTTATTTGATGCCTCTAGACATATGTTAGGCCGACGCA | SEQ ID NO: 87 |
| | AGCAGCCCGCGTGCG | |
| DR64-8452-F | TTAAAGAGGAGAAAGGTACCATGAGCCAGTTTGCGAACTA | SEQ ID NO: 88 |
| DR64-8452-R | TTTTATTTGATGCCTCTAGATTAAACCGCGCCCGGACTCA | SEQ ID NO: 89 |
| HisDR64-8447-F | GCATCACCATCACCATCACGGATCCATGTCGTACGCAATCT | SEQ ID NO: 90 |
| | ATCCCAGCC | |
| HisDR64-8447-R | TTTCGTTTTATTTGATGCCTCTAGATTATTCTCCGTACCAC | SEQ ID NO: 91 |
| | CCGGCGTCG | |
| HisDR64-8449-F | GCATCACCATCACCATCACGGATCCATGTCAGCATCCACAC | SEQ ID NO: 92 |
| | CGCGCCGGC | |
| HisDR64-8449-R | TTTCGTTTTATTTGATGCCTCTAGATTAGTGCGAATGCCTC | SEQ ID NO: 93 |
| | GGATTGCCG | |
| HisD 64-8450-F | GCATCACCATCACCATCACGGATCCATGTCCGCAACTTCTC | SEQ ID NO: 94 |
| | CATCCAGTT | |
| HisDR64-8450-R | TTTCGTTTTATTTGATGCCTCTAGATTAGGCCGACGCAAGC | SEQ ID NO: 95 |
| | AGCCCGCGT | |
| Bamb4925-Acc- | ACTGACCGAATTCATTAAAGAGGAGAAAGGTACCATGTCGG | SEQ ID NO: 96 |
| F | CAACAAAACCCAGGCTGCG | |
| Bamb4925-Nhe- | CTGCTCGATGTCATAGTTAATTTCTCCTGCTAGCTCAATGC | SEQ ID NO: 97 |
| R | GAATGGCTCGGCACGTCCG | |
| Bamb4923-Nhe- | GCCATTCGCATTGAGCTAGCAGGAGAAATTAACTATGACAT | SEQ ID NO: 98 |
| F | CGAGCAGCACACCGCGCTA | |
| Bamb4922-Hind- | CAATCTGTTGCATGGGTTTTTTCTCCTGAAGCTTTCAGCGG | SEQ ID NO: 99 |
| R | CCGAACGCGTCGGTCCCGA | |
| Bamb4918-Hind- | GTTCGGCCGCTGAAAGCTTCAGGAGAAAAAACCCATGCAAC | SEQ ID NO: 100 |
| F | AGATTGATCCGGCCGCGTC | |
| Bamb4918-Nde- | TCGTTTTATTTGATGCCTCTAGAGCTCACATATGTCAGCCG | SEQ ID NO: 101 |
| R | CGCGGCGCGCCCATGAATC | |
| Bamh4915-Acc- | ACTGACCGAATTCATTAAAGAGGAGAAAGGTACCATGACCG | SEQ ID NO: 102 |
| F | ACAGACGGATGCTGATCGC | |
| Bamb4915-Xba- | GACTGAGCCTTTCGTTTTATTTGATGCCTCTAGATCATATC | SEQ ID NO: 103 |
| R | GGGTAATGCCGCGGCGTGG | |
| BTH1632-Acc-F | ACCGAATTCATTAAAGAGGAGAAACCACGGTACCATGTCGG | SEQ ID NO: 104 |
| | CATCGAAACCCAAGCTGCG | |
| BTH1632- | GGCTCGTATTCATGGGTTTTTTCTCCTGGGATCCTCAGTGC | SEQ ID NO: 105 |
| BamHR | GAATGCCGCGGCACCGCGG | |
| BTH1630- | GCATTCGCACTGAGGATCCCAGGAGAAAAAACCCATGAATA | SEQ ID NO: 106 |
| BamHF | CGAGCCGTTCGCCGCGCTA | |
| BTH1629-Hind- | TTCGATGATTCCATAGTTAATTTCTCCTAAGCTTTCAGCGC | SEQ ID NO: 107 |
| R | TGAAACGGGTCGGCCGCGA | |
| BTH1625-Hind- | CGTTTCAGCGCTGAAAGCTTAGGAGAAATTAACTATGGAAT | SEQ ID NO: 108 |
| F | CATCGAATCGGCCGGCGCG | |
| BTH1625-Bp-R | GTTTTATTTGATGCCTCTAGAGCTCAGCCATATGTCACGCG | SEQ ID NO: 109 |
| | TTGCGCGCGAGCGCGAACC | |
| BTH1631-Acc-F | ACTGACCGAATTCATTAAAGAGGAGAAAGGTACCATGAACG | SEQ ID NO: 110 |
| | GGCCCACGGGCGAACTCCT | |
| BTH1631-Xba-R | GACTGAGCCTTTCGTTTTATTTGATGCCTCTAGATCACGTT | SEQ ID NO: 111 |
| | CGCGCACCCGCGCTCGCCT | |

To construct plasmid pBDO-1, five fragments of xylB, xylC, xylD, and xylX were amplified from Caulobacter crescentus genomic DNA by using primer pairs of xylBAcc-F/xylBBamHI-R, xylCBamHI-F/xylCNhe-R, xylDNheI-F/xylDHind-R, xylXHind-F/xylXBlpRem-R and xylXBlpRem-F/xylXBlpI-R, and then the fragment of xylX was amplified with primer pairs of xylXHind-F/xylXBlpI-R by using xylX-1 and xylX-2 as template. The four fragments of xylB, xylC, xylD and xylX were digested with Acc65I/BamHI, BamHI/NheI, NheI/HindIII and HindIII/BlpI, and then ligated with linearized pZAlac vector (Zhang et al., 2011. ChemSusChem 4:1068-1070) digested with Acc65I and BlpI to form the plasmid, pBDO-1. To make the plasmid pBDO-2, the coding region of xylA was PCR amplified by oligos of CC0822Acc-F and CC0822Xba-R using genomic DNA of C. crescentus as template, and then this coding region was inserted into the site between Acc65I and XbaI of vector pZElac (Zhang et al., 2011. ChemSusChem 4:1068-1070) after digestion. To construct the plasmids pBDO-3 and pBDO-12, the gene fragments of araC (BmulJ 5323), araD (BmulJ 5321), araA (BmulJ 5320), araB (BmulJ 5316) and araE (BmulJ 5314) were amplified from Burkholderia multivorans genomic DNA using primer pairs of araC-Acc65I-F/araC-NheI-R, araD-NheI-F/araD-remBlpI-R, araD-remBlpI-F/araA-HindIII-R, araA-HindIII-F/araB-NdeI-R and araB-NdeI-F/araE-BlpI-R respectively. The two fragments of araD-araA were then used as templates for overlap PCR using primer pair araD-NheI-F/araA-HindIII-R. The fragments araC, araD-araA, araB and araE were double-digested with enzymes Acc65I/NheI, NheI/HindIII, HindIII/NdeI and NdeI/BlpI respectively and these were ligated with linearized pZA-lac vector digested with Acc65I/BlpI to form the plasmid pBDO-3. To construct pBDO-12, fragment araB was amplified from B. multivorans genomic DNA using different primer pair araB-HindIII-F/araB-BlpI-R and the resulting PCR product was digested with HindIII/BlpI. The fragments araC Acc65I/NheI digest, araD-araA NheI/HindIII digest and araB HindIII/BlpI digest were ligated with linearized pZA-lac vector digested with Acc65I and BlpI to construct pBDO-12. To make plasmid pBDO-4, one fragment of vector from pBDO-1 plasmid, one fragment of ycbC from Bacillus subtilis, two fragments of garD-1 and garD-2 from E. coli, and two more fragments from Pseudomonas putida KT2440, were amplified using primer pairs of pZA-F/pZAAcc-R, KdaB S-F/KdaB S-R, GarD-F/GarD-Acc-R, GarD-Acc-F/GarD-R, udh-F/udh-Bsa-R, and udh-Bsa-F/udh-R, respectively. These six fragments were assembled by the golden gate method (Engler et al., 2008. PLoS One 3e3647) to form plasmid pBDO-4. Four fragments of BFD, kivd, ipdC and yqhD were amplified from genomic DNA of Pseudomonas putida, Lactococcus lactis, Salmonella typhimurium and E. coli, respectively, by using primer pairs of BFDAcc-F/BFDSphI-R, KIVDAcc-F/KIVDSphI-R, IPDCAcc-F/IPDCSphI-R and YqhDSphI-F/YqhDXbaI-R, respectively. Kivd and yqhD were digested with Acc65I/SphI and SphI/XbaI, and then inserted into the corresponding site of pZElac to form plasmid pBDO-5. Kivd in pBDO-5 was replaced by ipdC and BFD to build plasmids, pBDO-6 and pBDO-7. Two fragments of adhA and adh6 were amplified from L. lactis and S. cerevisiae genomic DNA, respectively by using primer pairs adhA-SphI-F/adhA-XbaI-R and Adh6-SphI-F/Adh6-XbaI-R, another two fragments of yahK and yjgB were amplified from E. coli genomic DNA with primer pairs of yahK-SphI-F/yahK-XbaI-R and yjgB-SphI-F/yjgB-XbaI-R. These four fragments were used to replace yqhD in plasmid pBDO-5 to make plasmids of pBDO-8, pBDO-9, pBDO-10 and pBDO-11.

Twenty Kivd mutant fragments of S286Y-1, S286Y-2, S286L-1, S286L-2, S286F-1, S286F-2, V461I-1, V461I-2, V461L-1, V461L-2, V461M-1, V461M-2, 1465F-1, I465F-2, I465H-1, I465H-2, I465L-1, I465L-2, I465M-1 and I465M-2 were amplified from plasmid pBDO-5 by using primer pairs of KIVDAcc-F/S286Y-R, S286Y-F/KIVDSphI-R, KIVDAcc-F/S286L-R, S286L-F/KIVDSphI-R, KIVDAcc-F/S286F-R, S286F-F/KIVDSphI-R, KIVDAcc-F/V461I-R, V461I-F/KIVDSphI-R, KIVDAcc-F/V461L-R, V461L-F/KIVDSphI-R, KIVDAcc-F/V461M-R, V461M-F/KIVDSphI-R, KIVDAcc-F/I465F-R, I465F-F/KIVDSphI-R, KIVDAcc-F/I465H-R, I465H-F/KIVDSphI-R, KIVDAcc-F/I465L-R, I465L-F/KIVDSphI-R, KIVDAcc-F/I465M-R and I465M-F/KIVDSphI-R, respectively. Ten fragments of S286Y, S286L, S286F, V461I, V461L, V461M, I465F, I465H, I465L and I465M amplified with primers KIVDAcc-F, KIVDSphI-R by using PCR templates of S286Y-1 and S286Y-2, S286L-1 and S286L-2, S286F-1 and S286F-2, V461I-1 and V461I-2, V461L-1 and V461L-2, V461M-1 and V461M-2, I465F-1 and I465F-2, I465H-1 and I465H-2, I465L-1 and I465L-2, I465M-1 and I465M-2, replaced the wild type kivd of pBDO-5 to form plasmids of pBDO-13, pBDO-14, pBDO-15, pBDO-16, pBDO-17, pBDO-18, pBDO-19, pBDO-20, pBDO-21 and pBDO-22.

To construct the plasmid pBDO-23, the gene fragments of DR64-8447, DR64-8448, DR64-8449 and DR64-8450 were amplified from Burkholderia xenovorans LB400 genomic DNA using primer pairs of DR64-8447-F/DR64-8447-R, DR64-8448-F/DR64-8448-R, DR64-8449-F/DR64-8449-R and DR64-8450-F/DR64-8450-R respectively. The fragments DR64-8447, DR64-8448, DR64-8449 and DR64-8450 were double-digested with enzymes Acc65I/NheI, NheI/HindIII, HindIII/NdeI and NdeI/BlpI respectively and these were ligated with linearized pZA-lac vector digested with Acc65I/BlpI to form the plasmid pBDO-23. To make the plasmid pBDO-24, the DR64-8452 gene was PCR amplified by oligos DR64-8452-F and DR64-8452-R using genomic DNA of B. xenovorans as template, and then this coding region was inserted into the site between Acc65I and XbaI of vector pZElac (Zhang et al., 2011. ChemSusChem 4:1068-1070) after digestion. To construct the plasmid pBDO-25, the gene fragments of Bamb4925, Bamb4923, Bamb4922 and Bamb4918 were amplified from Burkholderia ambifaria genomic DNA using primer pairs of Bamb4925-Acc-F/Bamb4925-Nhe-R, Bamb4923-Nhe-F/Bamb4922-HindR, and Bamb4918-Hind-F/Bamb4918-Nde-R respectively. The fragments Bamb4925, Bamb4923-4922 and Bamb4918 were double-digested with enzymes Acc65I/NheI, NheI/HindIII and HindIII/NdeI respectively and these were ligated with linearized pZA-lac vector digested with Acc65I/NdeI to form the plasmid pBDO-25. To make the plasmid pBDO-26, the Bamb4915 gene was PCR amplified by oligos Bamb4915-Acc-F/Bamb4915-Xba-R using genomic DNA of B. ambifaria as template, and then this coding region was inserted into the site between Acc65I and XbaI of vector pZElac (Zhang et al., 2011. ChemSusChem 4:1068-1070) after digestion. To construct the plasmid pBDO-27, the gene fragments of BTH_II1632, BTH_II1630, BTH_II1629 and BTH_II1625 were amplified from Burkholderia thailandensis genomic DNA using primer pairs of BTH1632-Acc-F/BTH1632-BamHR, BTH1630BamHF/BTH1629-Hind-R, and BTH1625-Hind-F/BTH1625-Blp-R respectively. The fragments BTH_II1632, BTH_II1630-1629 and BTH_111625 were double-digested with enzymes Acc65I/BamHI, BamHI/HindIII and HindIII/BlpI respectively and these were ligated with linearized pZA-lac vector digested with Acc65I/BlpI to form the plasmid pBDO-27. To make the plasmid pBDO-28, the BTH_II1631 gene was PCR amplified by oligos BTH1631-Acc-F/BTH1631-Xba-R using genomic DNA of B. thailandensis as template, and then this coding region was inserted into the site between Acc65I and XbaI of vector pZElac (Zhang et al., 2011. ChemSusChem 4:1068-1070) after digestion.

### Growth Assay

For the D-xylose, L-arabinose and D-galacturonate growth assays, the Δicd strains (BDO04 for D-xylose, BDO06 for L-arabinose and BDO08 for galacturonate) were transformed with 2-ketoglutarate producing plasmids (pBDO-1 and pBDO-2 for C. crescentus D-xylose, pBDO-3 for B. multivorans L-arabinose, pBDO-2 and pBDO-4 for D-galacturonate, pBDO-23 and pBDO-24 for B. xenovorans D-xylose, pBDO-25 and pBDO-26 for B. ambifaria L-arabinose, pBDO-27 and pBDO-28 for B. thailandensis L-arabinose). Three freshly transformed colonies were inoculated overnight in 2 ml 2×YT containing appropriate antibiotics. The optical density (OD) of all strains were measured using a spectrophotometer at 600 nm and the cell densities were normalized before starting the assays. M9 minimal media containing 5 g/l of each carbon source (glucose and D-xylose/L-arabinose/D-galacturonate), appropriate antibiotics and 0.2 mM IPTG was used for all assays. Optical density was measured every few hours using a spectrophotometer.

### Protein Expression and Purification

His-tagged plasmids were transformed into BL21 strain. The transformed cells were inoculated from an overnight pre-culture at 1/100 dilution and grown in 200 ml of 2×YT medium containing 100 mg/l ampicillin. When the OD₆₀₀ reached 0.6, 0.5 mM IPTG was added to induce protein expression, followed by incubation at 30° C. overnight. Then the cells were pelleted by centrifuging at 3,220 rcf for 15 minutes. The supernatants were discarded and the pellets were stored at −80° C. All the following steps were carried out at 4° C. to prevent protein degradation. For lysis, the cell pellets were first thawed on ice-water mixture and re-suspended in 15 ml lysis buffer. The lysis buffer (pH=7.6) contained 50 mM Tris-HCl, 100 mM NaCl, 10 mM imidazole, 5% glycerol, 1 mM DTT. Cell lysis was performed by sonication in a continuous mode set at 50% duty cycle and output control 5 (Heat Systems W-225). Each sample was sonicated for six cycles of one-minute sonication with intermittent one-minute cooling on ice-water mixture. The cell lysates were centrifuged at 10,733 rcf for 15 minutes. The supernatant was collected for purification. 4 ml of HisPur Ni-NTA resin solution (Thermo Fisher Scientific, Waltham, Mass.) was loaded in a column and the storage buffer was allowed to pass through by gravity to get a 2 ml final resin bed volume. The resin was equilibrated with 10 ml of lysis buffer and drained. The supernatant was then loaded in the column and allowed to pass through by gravity. The column was then washed twice with 10 ml of wash buffer (50 mM Tris-HCl, 100 mM NaCl, and 25 mM imidazole, pH=7.6). The bound protein was eluted with 15 ml of elution buffer (pH=8.0), which contained 50 mM Tris-HCl, 250 mMNaCl, and 250 mM imidazole. The final protein sample was then buffer-exchanged using AMICON ultra centrifugal filters (EMDMillipore, Billerica, Mass.) with the storage buffer (50 M Tris-HCl, 2 mM MgSO₄, 20% glycerol, pH=8.0). The concentrated protein were aliquoted (50 μl) into PCR tubes, flash frozen with dry ice and ethanol mixture and stored at −80° C. Purified protein concentration was determined by Quick Start Bradford protein assay kit (Bio-Rad Laboratories, Inc., Hercules, Calif.).

### Enzymatic Activity Assays

D-Xylose dehydrogenase (XylB/DR64-8447), L-arabinose dehydrogenase (AraA) and uronate dehydrogenase (Udh): Enzyme activities of XylB/DR64-8447, AraA and Udh were assayed by monitoring initial NADH generation at 340 nm and 30° C. using D-xylose, L-arabinose and D-galacturonate, as substrates respectively. Kinetic assays were carried out using 0 to 10 mM D-xylose/L-arabinose/D-galacturonate and 1 mM NAD⁺ in 100 mM Tris-HCl and 5 mM MgCl₂, pH 7.5. A series of enzymatic assays were conducted to estimate the initial activity as a function of starting substrate concentration. This data was used to fit the parameters of the Michaelis-Menten kinetic model, k_{cat} and Kₘ, by nonlinear least-squares regression using the intrinsic nlinfit function of the Matlab software program (Mathworks, Natick, Mass.). Kinetic constants (k_{cat}/Kₘ) for following enzymes were calculated with the same method.

Xylonate dehydratase (XylD/DR64-8449), L-arabonate dehydratase (AraC) and D-galactarate dehydratase (GarD): Enzyme activities of the three dehydratases were assayed according to a modified procedure of MacGee and Doudoroff using the semicarbazide method (Watanabe et al., 2006. J. Biol. Chem. 281:33521-33536). Kinetic assays were carried out using 100 nM of D-xylonate dehydratase/L-arabonate dehydratase/D-galactarate dehydratase in 100 mM Tris-HCl and 5 mM MgSO₄, pH 7.5. The reaction was initiated by adding D-xylonate/L-arabonate/D-galactarate and stopped after 0, 1 minute, 2 minutes, 3 minutes, 5 minutes, or 10 minutes with 2% (v/v) trifluoroacetic acid. The samples were then mixed with 100 μl of 0.1 M semicarbazide hydrochloride (containing 1.5% sodium acetate trihydrate) and incubated at room temperature for 30 minutes. Finally, the 2-ketoacids produced were quantified by detecting their semicarbazone absorbance at 250 nm.

2-Keto-3-deoxy-D-xylonate dehydratase (XylX/DR64-8450), 2-keto-3-deoxy-L-arabonate dehydratase (AraD) and 5-keto-4-deoxy-D-glucarate dehydratase (YcbC): Enzyme activities of XylX, DR64-8450, AraD and YcbC were assayed spectrophotometrically in a coupled assay with the corresponding previous dehydratase and 2-ketoglutaric semialdehyde dehydrogenase (KGSADH) as previously described (Watanabe et al., 2006. J Biol. Chem. 281:33521-33536). The assay was performed in 100 mM Tris-HCl buffer (pH-7.5) with 5 mM MgSO₄ containing 0-20 mM D-xylonate/L-arabonate/D-galactarate and 1 mM NAD⁺. After adding 100 nM D-xylonate dehydratase (XylD/DR64-8449)/L-arabonate dehydratase (AraC)/D-galactarate dehydratase (GarD) and 100 nM of the KGSADH, the mixture was incubated at 25° C. for 15 minutes. No change in absorbance at 340 nm was observed in this stage. The reaction was initiated by adding an appropriate amount of 2-keto-3-deoxy-D-xylonate dehydratase (XylX/DR64-8450)/2-keto-3-deoxy-L-arabonate dehydratase (AraD)/5-keto-4-deoxy-D-glucarate dehydratase (YcbC), and the increasing absorbance at 340 nm caused by NADH produced in the reaction was monitored.

2-Ketoacid decarboxylase (Kivd) and alcohol dehydrogenase (YqhD): The decarboxylase activity of Kivd was measured by a coupled enzymatic assay with AraC, AraD, and YqhD (Zhang et al., 2011. ChemSusChem 4:1068-1070). Excess AraC, AraD, and YqhD was used and the oxidation of NADPH was monitored at 340 nm. The assay mixture contained 1 mM NADPH, 1 μM AraC, 1 μM AraD, and 100 nM YqhD and 0.1-10 mM L-arabonate in assay buffer (100 mM Tris-HCl buffer, pH 7.5, 5 mM MgSO₄, 0.5 mM ThDP) with a total volume of 0.1 ml. The mixture was incubated at 30° C. for one hour and 10 nM Kivd was added. The dehydrogenase activity of YqhD was assayed according to NADPH initial consumption rates in a coupled assay. The assay mixture contained 1 mM NADPH, 100 nM Kivd, 1 μM AraC, 1 μM AraD, and 0.1-10 mM L-arabonate in 100 μl of 100 mM Tris-HCl buffer (pH 7.5) with 5 mM MgSO₄. The mixture was first incubated at 30° C. for one hour. Afterwards, 10 nM YqhD was added and the NADPH consumption rate was monitored.

### Shake Flask Batch Fermentation

125 ml conical flasks with 0.2 g CaCO₃ were autoclaved and dried to perform all small-scale fermentations. The flasks were filled with 5 ml M9 medium supplemented with 5 g/l yeast extract, 20 g/l glucose, 20 g/l D-xylose/L-arabinose/D-galacturonate and the corresponding antibiotics. 200 μl of overnight cultures incubated in 2×YT medium were transferred into the flasks and placed in a shaker at a speed of 250 rpm. After adding 0.1 mM isopropyl-β-D-thiogalactoside (IPTG), the flasks were sealed by parafilm and the fermentation was performed for 48 hours at 30° C. The fermentation products were measured by HPLC.

### Fed-Batch Fermentation in Bioreactor

Fermentation media for bioreactor cultures contained the following composition, in grams per liter: glucose, 10; yeast extract, 10; K₂HPO₄, 7.5; citric acid monohydrate, 2.0; yeast extract, 10; MgSO₄.7H₂O, 2.0, ferric ammonium citrate, 0.3; thiamine hydrochloride, 0.008; D-(+)-biotin, 0.008; nicotinic acid, 0.008; pyridoxine, 0.032; ampicillin, 0.1; kanamycin, 0.05; concentrated H₂SO₄, 0.8 mL; and 1 ml trace metal solution. Trace metal solution contained, in grams per liter: NaCl, 10; citric acid, 40; ZnSO₄.7H₂O, 1.0; MnSO₄.H₂O, 30; CuSO₄.5H₂O, 0.1; H₃BO₃, 0.1; Na₂MoO₄.2H₂O, 0.1; FeSO₄.7H₂O, 1.0; CoCl₂.6H₂O, 1.0. The feed solution contained, in grams per liter: glucose, 600; K₂HPO₄, 7.4; antifoam, 10 ml.

Fermentation experiments were performed in 1.3 L Bioflo 115 Bioreactors (Eppendorf AG, Hamburg, Germany) using an initial working volume of 0.5 L. The bioreactor was inoculated with 10% of overnight pre-culture with 2×YT medium. The culture condition was set at 37° C., 20% dissolved oxygen level (DO), and pH 6.8. After OD₆₀₀ reached 6.0, 0.2 mM IPTG and 20 g/l D-xylose, L-arabinose, or D-galacturonate was added. Temperature was changed to 30° C. and DO was set to 10%. The pH was controlled at 6.8 by automatic addition of 26% ammonium hydroxide. Air flow rate was maintained at 1 vvm during the whole process and DO was controlled by agitation rate (from 300 to 800 rpm). The feeding rate of glucose was manually adjusted according to the glucose consumption rate of cells to meet metabolic balance. D-xylose, L-arabinose, or D-galacturonate was added in batches. Fermentation culture was sampled every few hours to determine cell density and production level.

### Metabolite Analysis

Fermentation products were analyzed using an Agilent 1260 Infinity HPLC equipped with an Aminex HPX87H column and a refractive-index detector (RID). The mobile phase was 0.01 N H₂SO₄ with a flow rate of 0.6 ml/min. The column temperature and RID temperature were 35° C. and 50° C. respectively.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A recombinant cell modified to exhibit increased biosynthesis of 2,5-dioxopentanoate compared to a wild-type control.

2. The recombinant cell of claim 1 wherein biosynthesis of 2,5-dioxopentanoate includes nonphosphorylative metabolism.

3. The recombinant cell of claim 1 comprising a genetic modification to inhibit TCA cycle production of 2-ketoglutarate.

4. The recombinant cell of claim 3 wherein biosynthesis of 2,5-dioxopentanoate includes nonphosphorylative metabolism.

5. The recombinant cell of claim 2 wherein the genetic modification to inhibit TCA cycle production of 2-ketoglutarate comprises a genetic modification to decreased oxidation of isocitrate to 2-ketoglutarate compared to a wild-type control.

6. The recombinant cell of claim 3 wherein biosynthesis of 2,5-dioxopentanoate includes nonphosphorylative metabolism.

7. A method comprising:
providing the recombinant cell of claim 1;
introducing into the cell at least one heterologous polynucleotide that encodes an enzyme that catalyzes a nonphosphorylative metabolic step of biosynthesis of D-xylose; and
growing the cell in culture medium that comprises D-xylose.

8. The method of claim 7 further comprising measuring nonphosphorylative biosynthesis of D-xylose performed by the recombinant cell.

9. A method comprising:
providing the recombinant cell of claim 2;
introducing into the cell at least one heterologous polynucleotide that encodes an enzyme that catalyzes a nonphosphorylative metabolic step of biosynthesis of D-xylose; and
growing the cell in culture medium that comprises D-xylose.

10. The method of claim 9 further comprising measuring nonphosphorylative biosynthesis of D-xylose performed by the recombinant cell.

11. A method comprising:
providing the recombinant cell of claim 3;
introducing into the cell at least one heterologous polynucleotide that encodes an enzyme that catalyzes a nonphosphorylative metabolic step of biosynthesis of D-xylose; and
growing the cell in culture medium that comprises D-xylose.

12. The method of claim 11 further comprising measuring nonphosphorylative biosynthesis of D-xylose performed by the recombinant cell.

13. A method comprising:
providing the recombinant cell of claim 1;
introducing into the cell at least one heterologous polynucleotide that encodes an enzyme that catalyzes a nonphosphorylative metabolic step of biosynthesis of L-arabinose; and
growing the cell in culture medium that comprises L-arabinose.

14. The method of claim 13 further comprising measuring nonphosphorylative biosynthesis of L-arabinose performed by the cell.

15. A method comprising:
providing the recombinant cell of claim 2;
introducing into the cell at least one heterologous polynucleotide that encodes an enzyme that catalyzes a nonphosphorylative metabolic step of biosynthesis of L-arabinose; and
growing the cell in culture medium that comprises L-arabinose.

16. The method of claim 15 further comprising measuring nonphosphorylative biosynthesis of L-arabinose performed by the cell.

17. A method comprising:
providing the recombinant cell of claim 3;
introducing into the cell at least one heterologous polynucleotide that encodes an enzyme that catalyzes a nonphosphorylative metabolic step of biosynthesis of L-arabinose; and
growing the cell in culture medium that comprises L-arabinose.

18. The method of claim 17 further comprising measuring nonphosphorylative biosynthesis of L-arabinose performed by the cell.

19. A recombinant cell comprising:
at least one heterologous polynucleotide that encodes an enzyme in a nonphosphorylative biosynthetic pathway that converts D-xylose to 2,5-dioxopentanoate.

20. The recombinant cell of claim 19 wherein the enzyme comprises D-xylose dehydrogenase (XDH), D-xylonolactonase (XL), D-xylonate dehydratase (XD), or 2-keto-3-deoxy-D-xylonate dehydratase (KdxD).

21. The recombinant cell of claim 19 wherein the heterologous polynucleotide comprises a polynucleotide from a Burkholderia spp.

22. The recombinant cell of claim 21 wherein the heterologous polynucleotide comprises a B. xenovorans polynucleotide.

23. The recombinant cell of claim 22 wherein the B. xenovorans polynucleotide comprises one or more of: DR64_8447, DR64_8448, DR64_8449, DR64_8450, or DR64_8452.

24. The recombinant cell of claim 19 wherein the recombinant cell exhibits increased nonphosphorylative biosynthesis of D-xylose compared to a comparable recombinant cell comprising a polynucleotide that encodes a homologous nonphosphorylative biosynthetic pathway enzyme from Caulobacter crescentus.

25. A recombinant cell comprising:
at least one heterologous polynucleotide that encodes an enzyme in a nonphosphorylative biosynthetic pathway that converts L-arabinose to 2,5-dioxopentanoate.

26. The recombinant cell of claim 25 wherein the enzyme comprises L-arabinose dehydrogenase (ADH), L-arabinolactonase (AL), L-arabonate dehydratase (AD), and 2-keto-3-deoxy-L-arabonate dehydratase (KdaD).

27. The recombinant cell of claim 25 wherein the heterologous polynucleotide comprises a polynucleotide from a Burkholderia spp.

28. The recombinant cell of claim 27 wherein the heterologous polynucleotide comprises a B. xenovorans polynucleotide, a B. ambifaria polynucleotide, or a B. thailandensis polynucleotide.

29. The recombinant cell of claim 28 wherein the B. ambifaria polynucleotide comprises one or more of: Bamb_4925, Bamb_4924, Bamb_4923, Bamb_4922, Bamb_4921, Bamb_4920, Bamb_4919, or Bamb_4915.

30. The recombinant cell of claim 28 wherein the B. thailandensis polynucleotide comprises one or more of: BTH_II 1632, BTH_II 1631, BTH_II 1630, BTH_II 1629, BTH_II 1628, BTH-_II 1627, BTH_II 1626, or BTH_II 1625.

31. The recombinant cell of claim 25 wherein the recombinant cell exhibits increased nonphosphorylative biosynthesis of L-arabinose compared to a comparable recombinant cell comprising a polynucleotide that encodes a homologous nonphosphorylative biosynthetic pathway enzyme from Caulobacter crescentus.

32. A method comprising:
culturing a recombinant cell of claim 19 under conditions effective for the recombinant cell to exhibit increased biosynthesis of 2,5-dioxopentanoate compared to a wild-type control.

33. The method of claim 32 further comprising converting the 2,5-dioxopentanoate to glutamate, 1,4-butanediol, or glutaconate.

34. A method comprising:
culturing a recombinant cell of claim 25 under conditions effective for the recombinant cell to exhibit increased biosynthesis of 2,5-dioxopentanoate compared to a wild-type control.

35. The method of claim 34 further comprising converting the 2,5-dioxopentanoate to glutamate, 1,4-butanediol, or glutaconate.
